(19) United States
(12) Patent Application Publication
(10) Pub. No.: US 2023/0391867 A1
(43) Pub. Date: Dec. 7, 2023

(54) LONG ACTING BI-SPECIFIC T CELL ENGAGERS TARGETING CD3 AND CD47

(71) Applicant: Shenzhen Enduring Biotech, Ltd., Shenzhen (CN)

(72) Inventors: Yu Wen, Shenzhen (CN); Shumin Liu, Shenzhen (CN); Weidong Lyu, Shenzhen (CN); Dechun Wu, Shenzhen (CN)

(73) Assignee: Shenzhen Enduring Biotech, Ltd., Shenzhen (CN)

(21) Appl. No.: 18/031,927

(22) PCT Filed: Nov. 17, 2021
(86) PCT No.: PCT/CN2021/131181

§ 371 Date: Apr. 14, 2023

(30) Foreign Application Priority Data
Nov. 17, 2020 (WO) PCT/CN2020/129349

(51) Int. Cl. C07K 16/28; A61P 35/00; C07K 16/46

(52) U.S. Cl. CPC C07K 16/2803; C07K 16/2809; A61P 35/00; C07K 16/468; C07K 2317/52; C07K 2317/31; C07K 2317/622; C07K 2317/92; C07K 2317/732; A61K 2039/505

(57) ABSTRACT

Provided are bispecific molecules and, in particular, long acting bispecific T cell engagers targeting CD3 and CD47 with improved efficacy, toxic profile and therapeutic window, and methods of making and using such long acting bispecific binding molecules.

This application is the U.S. national phase of International Patent Application No. PCT/CN2021/131181 filed on Nov. 17, 2021 which claims the benefit of International Patent Application No.: PCT/CN2020/129349 filed on Nov. 17, 2020, the entire contents of which are incorporated by reference for all purpose.

### REFERENCE TO A SEQUENCE LISTING

This application incorporates by reference the Sequence Listing submitted in Computer Readable Form as file F21W0106-PCT-SL-20211116, created on Nov. 16, 2021 and containing 9,129 bytes.

### FIELD OF INVENTION

The present invention relates to bispecific molecules and, in particular, relates to long acting bispecific T cell engagers targeting CD3 and CD47 with improved efficacy, toxic profile and therapeutic window, and also relates to methods of making and using such long acting bispecific binding molecules.

### BACKGROUND OF INVENTION

Emerging research data from cancer immuno-oncology demonstrates that augmenting antitumor immune responses offer great opportunity to potentiate durable remission in cancer (Decker, W. K., etal., 2017, Front Immunol, 8, p829; Queudeville, M., etal., 2017, Onco Targets Ther. 10, p3567-35′78). The Bi-specific T-cell engager (BiTE) Blinatumomab (Blincyto®) is one of those new immuno-oncology agents that provides high efficacy for relapsed or refractory (R/R) B lineage leukemia or lymphoma (Aldoss, I., et al., 2017, Leukemia, 31(4), p777-787). Blinatumomab is a fusion protein of two single-chain antibodies linked by a small peptide chain with dual affinity for CD19 and CD3. The simultaneous binding to both CD3-expressing T cells and CD19-expressing malignant B cells activates and engages wide spectrum of cytotoxic T cells to Blinatumomab-bound malignant B cells, resulting in the lysis of target CD19⁺ B cancerous cells (Yuraszeck, T., et al., 2017, Clin Pharmacol Ther, 101(5), p634-645).

However, similar to other recombinant proteins, Blinatumomab is cleared very quickly from blood circulation (t_{1/2}=1.25 hour in patients), which limits its application in other types of cancers such as solid tumors because of the short half-life. To maintain a certain minimal level of the drug in blood, Blinatumomab is required to be administered by a continuous intravenous infusion for 4 weeks (24 hours a day, 7 days a week) with a portable mini-pump. This type of drug administration possesses a great burden for patients to comply with, particularly for young children (Portell, C. A., et al., 2013, Clin Pharmacol, 5(Suppl 1), p5-11). As a result, infection has been one of the main adverse events in administering Blinatumomab to patients (Wilke, A.C. et al., 2017, Expert Opin Drug Saf., 16(10), p1191-1202) and high chance of infection or even life threatening infection has put these patients at great risk.

In addition, although Blinatumomab is highly efficient in killing tumor cells, the same powerful mechanism also limits its application to other types of diseases such as B cell associated autoimmune diseases because of severe cytokine storm accompanied with the treatment that might endanger patient's life.

Another important field of immune-oncology is immune-checkpoint. For the last decade, immune checkpoint blockade has been a focused category of immunotherapy. Encouraged by regulatory approved “adaptive immune checkpoint blockades” such as anti-PD1, anti-PDL1 therapies etc. and their market successes, chasing of “innate immune checkpoint blockades” that target CD47 or its receptor signal regulatory protein a (SIRPa) becomes ferocious. By targeting CD47/SIRP, phagocytes opsonized by tumor specific antibodies toward phagocytosis of tumor cells could be re-invigorated^{1,2}. A number of early phase clinical trials have already demonstrated promising therapeutic efficacy (Sikic, B. I., et al., 2019, J Clin Oncol, 37(12), p946-953; Sallman, D. A., et al., 2019, Journal of Clinical Oncology, 37(15 suppl), p7009-'7009; Ansell, S., et al., 2016, Blood, 128(22), p1812-1812). However, certain types of normal cells such as red blood cells, T cells, NK cells and platelets also express high levels of CD47 comparable to those expressed in tumor cells (Oldenborg, P. A., et al., 2000, Science, 288(5473) p2051-4; Strizova, Z., et al., Scientific reports, 2020. 10(1), p13936-13936; Velliquette, R. W., et al., 2019, Transfusion, 59(2), p730-737). Therefore, treatment with anti-CD47 antibody frequently result in anti-CD47 induced anemia, lymphopenia and thrombocytopenia due to its Fc component induced ADCP. Even though theses adverse events might be alleviated through proper dose management, lymphopenia caused T cell death could negatively influence the efficacies of the CD47 blockades. This is one of the reasons that the ratio of patients responding to the CD47 blockade immunotherapies are not as high as the novel mechanism could potentially provide.

Therefore, there is a need to develop more efficient and safer innate immune checkpoint blockade therapy targeting CD47.

### SUMMARY OF THE INVENTION

In this invention, a novel anti-CD3Xanti-CD47 BiTE molecule is developed which improves the efficacy of the therapy targeting innate immune checkpoint molecule CD47 by its dual action of mechanisms: blocking innate immune checkpoint signaling pathway and redirecting cytotoxic T cells to tumor cells so as to kill the cancer cells. Furthermore, unlike traditional anti-CD47 antibodies, the anti-CD3Xanti-CD47 BiTE molecule of the present disclosure causes substantially no anemia, lymphopenia etc., and the molecule confers desired safety while retainning strong efficacy.

In one aspect, the invention provides a bi-specific molecule or compound having Formula Ia:

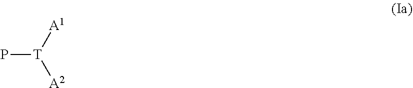

wherein:

P is a non-immunogenic polymer; T is a trifunctional linker moiety, e.g. a trifunctional small molecule linker moiety, and has one, two, or more functional groups that are capable of site-specific conjugation with one, two or more the same or different proteins or peptides; and one of A¹ and A² is an anti-CD3 antibody or an antigen binding fragment thereof, and the other is an anti-CD47 antibody or antigen binding fragment thereof, wherein the anti-CD3 antibody and/or the anti-CD47 antibody lack a functional Fc region, e.g. the Fc region is modified to abolish or diminish one or more of its effector functions including antibody-dependent cell-mediated cytotoxicity (ADCC), antibody-dependent cellular phagocytosis (ADCP) or complement dependent cytotoxicity (CDC).

In particular, an aspect of the invention provides a compound having Formula Ib:

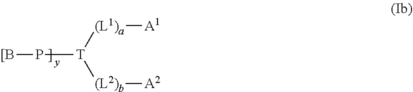

wherein:

P is a non-immunogenic polymer;

B is H or a capping group selected from C₁₋₁₀ alkyl and aryl, wherein one or more carbons of said alkyl or aryl is optionally replaced with a heteroatom;

One of A¹ and A² is an anti-CD3 antibody or an antigen binding fragment thereof, and the other is an anti-CD47 antibody or antigen binding fragment thereof, wherein the anti-CD3 antibody and the anti-CD47 antibody lack a functional Fc region, e.g. the Fc region is modified to abolish or diminish one or more of its effector functions including ADCC, ADCP or CDC;

L¹ and L² are each independently a bifunctional linker or a peptide;

a and b are each independently an integer selected from 1-10;

y is an integer selected from 1-10;

and

T is a trifunctional linker moiety comprising two linkages for (L¹)ₐ-A¹ and (L²)_{b}-A² and one linkage for P.

In some embodiments of the above compound having Formula (Ia) or Formula I(b), T is a tri-functional linker (e.g. an amino acid) having one, two, or more functional groups that, after derivatization and/or extension with a bifunctional spacer, are capable of site-specific conjugate with A¹ and A² or their derivatives consecutively, wherein the linkage between T and (L¹)ₐ and the linkage between T and (L²)_{b} could be same or different.

In some embodiments of the above compound having Formula (Ia) or Formula I(b), the anti-CD3 antibody and/or the anti-CD47 antibody lack the Fc region of a classic immunoglobulin molecule. In some embodiments, both the anti-CD3 antibody and the anti-CD47 antibody lack the Fc region.

In some embodiments, the anti-CD3 antibody and the anti-CD47 antibody are each independently selected from a Fab, a single chain antibody (e.g. a scFv), and a nanobody (a single domain antibody). In some embodiments, both the anti-CD3 antibody and the anti-CD47 antibody are single chain antibodies.

The anti-CD47 antibody may target any portion, peptide fragment or epitope of the full length CD47 (Barclay, A.N. et al., 2014, Annu Rev Immunol, 32, p25-50). The anti-CD3 antibody may target any portion, peptide fragment or epitope from any one of the subunits in the T cell receptor complex, namely CD3 gamma, CD3 delta, CD3 epsilon, CD3 zeta and CD3 eta (Kuhns, M.S., et al., 2006, Immunity, 24 (2), p133-9).

In some embodiments, the two linkages of T for (L¹)ₐ-A¹ and (L²)_{b}-A², the linkage between (L¹)ₐ and A¹, the linkage between (L²)_{b} and A² and the linkage within (L¹)ₐ or (L²)_{b} may be each independently derived from functional groups selected from the group consisting of alkyl halide, acid halide, aldehyde, ketone, ester, anhydride, carboxylic acid, amide, amine, hydrazide, alkylhydrazines, hydroxy, epoxide, thiol, maleimide, 2-pyridyldithio varian, aromatic or vinyl sulfone, acrylate, bromo or iodo acetamide, azide, alkene, alkyne, dibenzocyclooctyl (DBCO), 2-amino-benzaldehyde or 2-amino-acetophenone group, hydrazide, oxime, potassium acyltrifluoroborate, O-carbamoylhydroxylamine, trans-cyclooctene, tetrazine and triarylphosphine.

In some embodiments, L¹ and L² each may comprise a spacer independently selected from the group consisting of —(CH₂)ₘXY(CH₂)ₙ—, —X(CH₂)ₘO(CH₂CH₂O)p(CH₂)ₙY—, —(CH₂)ₘX—Y(CH₂)ₙ—, —(CH₂)ₘheterocyclyl-, —(CH₂)ₘX—, —X(CH₂)ₘY—, and an amino acid or peptide having 2 to 50 amino acid residues; wherein m, n, and p in each instance are independently an integer ranging from 0 to 25; X and Y in each instance are independently selected from the group consisting of C(═O), CR₁R₂, NR₃, S, O, or Null, wherein R₁ and R₂ independently represent hydrogen, C₁₋₁₀ alkyl or (CH₂)₁₋₁₀C(═O), R₃ is H or a C₁₋₁₀ alkyl, and wherein the heterocyclyl is derived from an maleimido, strained alkenes and alkynes, azide or a tetrazolyl moiety.

In some embodiments, the non-immunogenic polymer P may comprise polyethylene glycol (PEG), dextrans, carbohydrate-base polymers, polyalkylene oxide, polyvinyl alcohols, hydroxypropyl-methacrylamide (HPMA), or a co-polymer thereof. Preferably, the non-immunogenic polymer P may comprise PEG, such as a branched PEG or a linear PEG.

In some embodiments, at least one terminal of linear PEG or branch PEG is capped with H, methyl or low molecule weight alkyl group. The total molecule weight of the PEG may be 3,000 Da to 100,000 Da, e.g., 5,000 Da to 80,000 Da, 10,000 Da to 60,000 Da, and 20,000 Da to The PEG may be linked to tri-functional linker T moiety either through a permanent bond or a cleavable bond.

In some embodiments, the non-immunogenic polymer P may comprise PEG and B is methyl or a C₁₋₁₀ alkyl.

In some embodiments, the linkage of T to P may be cleavable.

In some embodiments, the linkage of T to P may be selected from the group consisting of amide, ester, carbamate, carbonate, imide, imine, hydrazones, sulfone, ether, thioether, thioester and disulfide.

In some embodiments, T may be derived from a natural or unnatural amino acid selected from the group consisting of cysteine, lysine, asparagine, aspartic, glutamic acid, glutamine, histidine, serine, threonine, tryptophan, tyrosine or genetically-encoded alkene lysine (such as N6-(hex-5-enoyl)-L-lysine), 2-Amino-8-oxononanoic acid, m- or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino) hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ϵ}-Acryloyl-1-lysine, Nϵ-5-norbornene-2-yloxycarbonyl-1-lysine, N-ϵ-(Cyclooct-2-yn-1-yloxy)carbonyl)-L-lysine, N-ϵ-(2-(Cyclooct-2-yn-1-yloxy)ethyl) carbonyl-L-lysine, and genetically encoded tetrazine amino acid (such as 4-(6-methyl-s-tetrazin-3-yl)aminophenylalanine).

In some embodiments, T may be derived from lysine or cysteine.

In some embodiments, the non-immunogenic polymer P may be derived from a PEG having a terminal maleimide or 2-pyridyldithio varian or aromatic sulfone or vinyl sulfone. T may be derived from cysteine, and the linkage between P and T may be a thioether or disulfide.

In some embodiments, the non-immunogenic polymer P may be derived from a PEG having a terminal maleimide, and (L¹)ₐ-T-(L²)_{b} may be a peptide having 3-100 amino acid residues, e.g. 3-50 amino acid residues.

In some embodiments, the anti-CD3 antibody may be an anti-CD3 single chain antibody (e.g. a scFv) and/or the anti-CD47 antibody may be an anti-CD47 single chain antibody (e.g. a scFv). In a preferred embodiment, both the anti-CD3 antibody and the anti-CD47 antibody are single chain antibodies (e.g. scFvs).

In some embodiments, (L¹)ₐ or (L²)_{b} may comprise a linkage formed from azide and alkyne or formed from maleimide and thiol. In some embodiments, the alkyne may be dibenzocyclooctyl (DBCO). In some embodiments, T is lysine, P is PEG, and y is 1, and the alkyne is dibenzocyclooctyl (DBCO). In some embodiments, one of A¹ and A² may be derived from an azide tagged antibody, antibody chain, antibody fragment, single chain antibody or a single domain antibody, wherein the azide is conjugated to an alkyne in the respective (L¹)ₐ or (L²)_{b}; the other of A¹ and A² may be derived from a thiol tagged antibody, antibody chain, antibody fragment, single chain antibody or a single domain antibody, wherein the thiol is conjugated to a maleimide in the respective (L¹)ₐ or (L²)_{b}.

In one aspect, the invention provides a compound having the following structure:

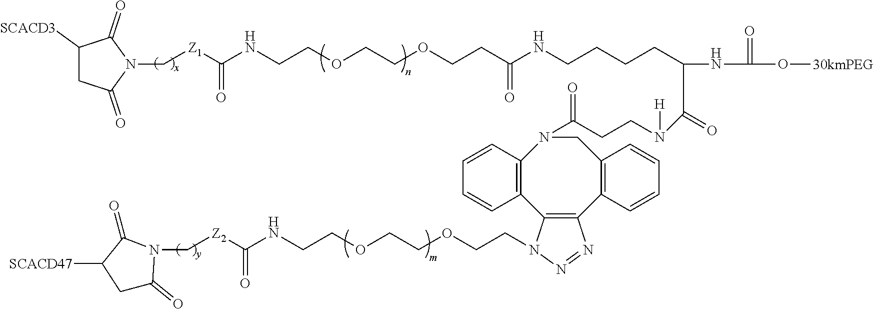

wherein SCACD3 is a single chain anti-CD3 antibody, SCACD47 is a single chain anti-CD47 antibody, Z1 and Z2 are each independently selected from CH₂, a low-molecular-weight alkane and cyclohexane or its derivative, and n, m, x and y are each independently an integer selected from 1-50, e.g. an integer selected from 1-10.

In another aspect, the invention provides a compound having the following structure:

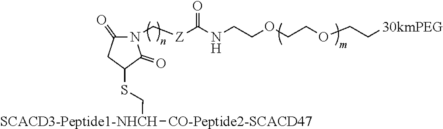

wherein SCACD3 is a single chain anti-CD3 antibody, SCACD47 is a single chain anti-CD47 antibody, Z is selected from CH₂, a low-molecular-weight alkane and cyclohexane or its derivative, and n and m are each independently an integer selected from 0-50, e.g. an integer selected from 0 -10. Peptide 1 and peptide 2 may be the same or different and wherein peptide 1 and peptide 2 each may independently comprises 2-50 amino acid residues.

In some embodiments of above aspects, the anti-CD3 antibody may comprise the amino acid sequence set forth in SEQ ID NO: 1.

In some embodiments of above aspects, the anti-CD47 antibody may comprise the amino acid sequence set forth in SEQ ID NO: 2.

In some embodiments of above aspects, the anti-CD3 antibody may comprise the amino acid sequence set forth in SEQ ID NO: 1, and the anti-CD47 antibody may comprise the amino acid sequence set forth in SEQ ID NO: 2.

In one aspect, the invention provides a method for preparing a compound of any of the above aspects.

In some embodiments, the method may comprise preparing a compound having the structure

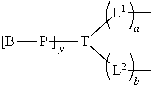

with two terminal functional groups capable of site-specific conjugation with two different proteins or modified versions thereof; and stepwise site-specific conjugating A¹ and A² to the terminal functional groups to form the compound of the Formula (Ib), wherein optionally one or both of A¹ and A² are modified before conjugation.

In other embodiments, the method may comprise preparing a compound having the structure

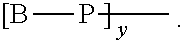

with a terminal functional group, and reacting

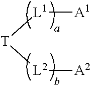

or a derivative thereof with the terminal functional group to form the compound of the Formula (Ib). In some embodiments, the (L¹)ₐ-T-(L²)_{b} may be a peptide having 3-100 amino acid residues, e.g. 3-50 amino acid residues.

In some embodiments, the above-described bi-specific molecule or compound may be prepared according to a method comprising: (i) preparing a non-immunogenic polymer with terminal bi-functional groups capable of site-specific conjugation with two different proteins, anti-CD3 and anti-CD47 (or their modified versions) respectively; and (ii) stepwise site-specific conjugating the non-immunogenic polymer with anti-CD3 and anti-CD47 or their modified versions to form a compound of Formula Ia or Ib. In some embodiments, before the preparation step, the proteins may be modified with a small molecule linker first. Alternatively, the above-described bi-specific molecule or compound might be made according to a method comprising: preparing a fusion anti-CD3 and anti-CD47 protein with a thiol tag followed by PEGylation with thiol specific PEG reagent such as PEG maleimide, PEG 2-pyridyldithio varian, or PEG aromatic or vinyl sulfone.

In one aspect, the invention provides a conjugate (e.g. an antibody-drug conjugate) comprising the bi-specific molecule or compound of the invention and one or more effector moieties conjugated to the molecule or compound.

In some embodiments, the one or more effector moieties may be selected from the group consisting of cytotoxic agents, chemotherapeutic agents, growth inhibitory agents, toxins, and radioactive isotopes.

The invention also provides a pharmaceutical formulation comprising the bi-specific molecule or compound of the invention and optionally a pharmaceutically acceptable carrier.

In some embodiments, the pharmaceutical composition may further comprise an additional therapeutic agent. In some embodiments, the additional therapeutic agent may be selected from the group consisting of a cytotoxic agent, a chemotherapeutic agent, an antibody, an antibody drug conjugate, and a small molecule drug.

The invention further provides a method of treating a disease in a subject in need thereof comprising administering an effective amount of the bi-specific molecule or compound, conjugate and/or the phramaceutical composition of the invention.

In one aspect, the invention provides use of the bi-specific molecule or compound, conjugate, or the phramaceutical composition of the invention in the manufacture of a medicament for treating a disease in a subject in need thereof.

In another aspect, the invention provides the the bi-specific molecule or compound, conjugate, or the phramaceutical composition of the invention for use in treating a disease in a subject in need thereof.

In some embodiments of the above method, use, and bi-specific molecule or compound, conjugate or the phramaceutical composition for use, the disease may be a cancer, e.g. a cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer.

The details of one or more embodiments of the invention are set forth in the description below. Other features, objectives, and advantages of the invention will be apparent from the description and from the claims.

### BRIEF DESCRIPTION OF THE DRAWINGS

FIG. 1. Schematic graphics illustrating the reaction scheme of preparing 30kmPEG-Lys(maleimide)-DBCO as described in Example 1;

FIG. 2. Schematic graphics illustrating the reaction scheme of preparing PEGylated single chain antibody 30kmPEG-(SCACD3)SCACD47 as described in Example 3 and as described in Example 5;

FIG. 3: Results of in-vitro T cell mediated cytotoxicity as described in Example 6;

FIG. 4: Results of in vitro hemagglutination experiment as described in Example 7;

FIG. 5: Results of detection of apoptotic T cells induced by JY102 with flow cytometry as described in Example 8;

FIG. 6: Results of JY102 binding to target cells expressing CD47 with flow cytometry as described in Example 9;

FIG. 7: Results of JY102 and JY102-BiTE binding to human CD3 protein in ELISA assay as described Example 10;

FIG. 8: Results of JY102 and JY102-BiTE binding to human CD47 protein in ELISA assay as described in Example 10;

FIG. 9: Results of in vivo phamacokinetcis study of JY102 as described in Example 11.

FIG. 10: Results of preliminary toxicity evalutaion of JY102 on PBMC humanized mice as described in Example 12;

FIG. 11: Results of in vivo efficacy of tumor growth inhibition of JY102 as described in Example 13.

### DETAILED DESCRIPTION OF THE INVENTION

Cancer can be seen as the consequence of tumorous cells escaping from immunosurveillance. Manipulation of human immune system to re-engage cytotoxic T cells to kill cancer has been greatly appreciated in the last two decades exemplified with the development of BiTE prototype compound Blinatumomab, which shows high efficiency in treatment of patients with hematologic tumors and was approved by FDA as the first BiTE bispecific antibody.

BiTE bispecific antibody can activate T cells directly through the CD3 complex which is downstream of TCR (T cell receptor) on the T cell activation pathway. The function of BiTEs is independent of T cell receptor specificity, MHC restriction, and costimulatory signals. Typical BiTEs are relatively small in size (MW is about ˜55 kD) which allows their two arms effectively bridging T cells to targeted cells to form an immunological synapse. The formation of an immunological synapse favors T cell activation and cytotoxic effect for killing tumor cells through a granzyme and perforin-mediated process, which is a common mechanism to all cytotoxic T cells activated by antigens conventionally. However, unlike the conventional T cell activation mechanism, T cells activation by BiTE does not require a co-stimulative signal. In addition, the anti-CD3 portion of BiTE activates the T cells downstream of the TCR/CD3 complex directly, bypassing the antigen specificity required by TCR. Therefore, theoretically all T cells could be activated by BiTE.

Blinatumomab is a product of BiTE prototype. It is bispecific fusion antibody, composed of two single-chain monoclonal antibodies against CD19 and CD3. Similar to other small recombinant proteins, Blinatumomab is cleared very quickly during blood circulation (t_{1/2}=1.25 hour in patients). To maintain a certain minimal level of the drug in blood, Blinatumomab is required to be administered by a continuous intravenous infusion for 4 weeks (24 hours a day, 7 days a week) with a portable mini-pump (Portell, C. A. et al., 2013, Clin Pharmacol, 5 (Suppl 1), p5-11). This type of drug administration possesses a great burden for patients to comply with, particularly for young children. Additionally, high chance of infection or even life threatening infection has put these patients at great risk.

Moreover, due to their short half-life, fusion single chain bispecific antibodies typically have poor retention time, which limits their applications in other types of cancers such as solid tumors.

Over the last decade or so, immune checkpoint blockade has been a focused category of immunotherapy for cancer treatment. Several “adaptive immune checkpoint blockades” such as anti-PD1, anti-PDL1 etc. have been approved and successfully marketed.

CD47 is a component of innate immune checkpoint on tumor cells and functions as a “do not eat me” signal through interacting with its receptor signal regulatory protein alpha (SIRPa) on professional phagocytic cells (e.g. macrophage and neutrophil). CD47 is overexpressed on tumor cells in almost all cancer types (Willingham, S. B., et al., 2012. Proc Natl Acad Sci U S A. 109(17), p6662-'7; Chao, M. P., et al., 2012, Current opinion in immunology, 24(2), p225-232) . The overexpression of CD47 is associated with poor prognosis or recurrence in clinic testing (Chan, K. S., et al., 2009, Proc Natl Acad Sci U S A, 106(33), p14016-21; Yuan, J., et al., 2019, Oncol Lett, 18(3), p3249-3255; Majeti, R., et al., 2009, Cell, 138(2), p286-99). In tumors, CD47 is exploited by tumor cells as an antiphagocytic ligand to blunt antibody (neoantigen specific) effector functions by transmitting an inhibitory signal through its receptor SIRPa on phagocytic cells. Therefore, elevated CD47 levels are associated with immune escape in cancers.

Although CD47 is broadly expressed at low levels on normal cells, the high expression levels of CD47 in certain types of normal cells, such as T cells, NK, red blood cells, and platelets and the like (Strizova, Z., et al., 2020, Scientific reports, 10(1), p13936-13936; Olsson, M., et al., 2005, Blood, 105(9), p3577-82) have brought huge challenge for developing CD47/SIRPa blocking immunotherapeutic agents. In three phase I clinical trials, it is reported that treatments with anti-CD47 antibody induced anemia in up to 57% patients. Thrombocytopenia and lymphopenia were also frequently observed adverse events. These events are all directly related to the phagocytic cytotoxicity due to the high expression of CD47 in these normal cells. It is reported that, in an animal model study, durable antitumor response to CD47 blockade requires adaptive immune stimulation (Sockolosky, J. T., et al., 2016, Proceedings of the National Academy of Sciences of the United States of America, 113(19), pE2646-E2654), and therefore lymphopenia caused T cell decrease would negatively influence the clinical efficacy, as potential phagocytes of T cells is contradictory to the therapeutic goal and would weaken the rational basis for the design that combines anti-CD47 with anti-PD-1 or anti-PD-Ll. Indeed, it has been found that the presence of lymphopenia at the beginning of immunotherapy was related to inferior disease control and shorter survival in a retrospective analysis for NSCLC patients treated with anti-PD-1 or anti-PD-Ll (Galli, G., et al., 2018, Annals of Oncology, 29(suppl), pviii512).

In this invention, a novel anti-CD3Xanti-CD47 BiTE molecule is developed which improves the efficacy of the therapy targeting innate immune checkpoint molecule by its dual action of mechanisms: blocking innate immune checkpoint signaling and redirecting cytotoxic T cell to tumor cells to kill the cancer cells. Furthermore, unlike traditional anti-CD47 monoclonal antibodies, the anti-CD3Xanti-CD47 BiTE molecule causes substantially no anemia, lymphopenia etc., and the molecule confers desired safety while retains strong efficacy.

Accordingly, this invention addresses the above discussed problems and improves current anti-CD47 antibody therapy.

### Conjugates

In one aspect, the invention provides a bi-specific molecule, conjugate, or compound having Formula Ia,

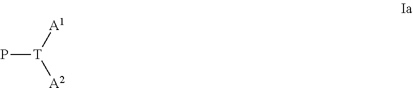

wherein:

P is a non-immunogenic polymer; T is a trifunctional linker moiety, e.g. a trifunctional small molecule linker moiety, and has one, two, or more functional groups that are capable of site-specific conjugation with one, two or more the same or different proteins or peptides; and one of A¹ and A² is an anti-CD3 antibody or an antigen binding fragment thereof, and the other is an anti-CD47 antibody or antigen binding fragment thereof, wherein the anti-CD3 antibody and/or the anti-CD47 antibody lack a functional Fc region, e.g. the Fc region is modified to abolish or diminish one or more of its effector functions including ADCC, ADCP or CDC.

In particular, an aspect of the invention provides a compound having Formula Ib:

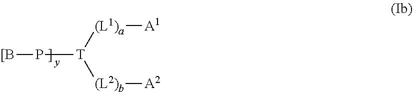

wherein:

P is a non-immunogenic polymer;

B is H or a capping group selected from C1-10 alkyl and aryl, wherein one or more carbons of said alkyl or aryl is optionally replaced with a heteroatom;

One of A¹ and A² is an anti-CD3 antibody or an antigen binding fragment thereof, and the other is an anti-CD47 antibody or antigen binding fragment thereof, wherein the anti-CD3 antibody and the anti-CD47 antibody lack a functional Fc region, e.g. the Fc region is modified to abolish or diminish one or more of its effector functions including ADCC, ADCP or CDC;

L¹ and L² are each independently a bifunctional linker or a peptide;

a and b are each independently an integer selected from 1-10;

y is an integer selected from 1-10;

and

T is a trifunctional linker moiety comprising two linkages for (L¹)ₐ-A¹ and (L²)_{b}-A² and one linkage for P.

The P moiety of the conjugate may be prepared from various non-imunogenic polymers. Preferably, the polymer is water-soluble. Examples of the polymers include polyethylene glycol (PEG), dextrans, carbohydrate-based polymers, polyalkylene oxide, polyvinyl alcohols and other non-immunogenic polymers. Further exemplary polymers include poly(alkyleneglycol), poly(olefinic alcohol), poly(vinylpyrrolidone), poly(hydroxyalkylmethacrylamide), poly(hydroxyalkylmethacrylate), poly(saccharide), poly(α-hydroxy acid), poly(acrylic acid), poly(vinyl alcohol), polyphosphazene, polyoxazoline, poly(N-acryloylmorpholine), or copolymers or terpolymers thereof. The polymers may be liner or branched.

In some embodiments, The total molecule weight of the polymer may range from 3,000 Da to 100,000 Da, e.g., 5,000 Da to 80,000 Da, 10,000 Da to 60,000 Da, and 20,000 Da to 40,000 Da, with all subranges included.

The polymer may comprise a terminus group capable of being functionalized, activated, or conjugated to a reaction partner. Non-limiting examples of the terminus groups include hydroxyl, amino, carboxyl, thiol, maleimide, azide, alkyne, DBCO and halide.

In some embodiments, the polymer comprises polyethylene glycol (PEG).

In some embodiments, B in [B-P]_{y} is a H or a low molecular weight C₁₋₁₀ alkyl group such as methyl, ethyl, and butyl, wherein one or more of the carbons may be replaced by a heteroatom (e.g. O, S, and N).

In some embodiments, y is 1 and Formula Ib represents a conjugate with a pendent polymer chain. In some embodiments, y is 2, 3, 4, 5 or 6 and Formula Ib represents a conjugate comprising a branched polymer moiety. In some embodiments, chemical bond between P and T is cleavable.

In some embodiments, P represents a PEG moiety. In some embodiments, methods of preparing terminal branched heterobifunctional PEG that is capable of site-specific conjugating with two different proteins, such as antibody fragments or single chain antibodies, are provided. In some embodiments, methods for preparing PEGylated bispecific single chain antibody that is able to extend blood circulation half life and improves toxic profiles thereof when treating with the compound are also provided.

In some exemplary embodiments, a terminal functional group of PEG such as hydroxyl or carboxyl group etc., is activated and conjugated with a trifunctional small molecule moiety such as Boc protected lysine to form a terminal branched heterobifunctional PEG-Lys(Boc)-OH. Deprotection of Boc gives PEG-lysine, which is then converted to PEG-Lys(Maleimide)-OH by reacting with a bifunctional small molecule spacer that has maleimide group. The carboxyl group of PEG-Lys(Maleimide)-OH is then converted to alkyne group by coupling with another bifunctional small molecule spacer that has alkyne group such as NH2-DBCO to form a terminal branched PEG-Lys(Maleimide)-DBCO. This terminal branched PEG-Lys(Maleimide)-DBCO is site-specifically conjugated with a thiol tagged single chain anti-CD3 and an azide tagged single chain anti-CD47 consecutively to form a PEGylated single chain bispecific antibody (PEG-(anti-CD3)ₐnti-CD47).

Alternatively, an anti-CD3-peptide-antiCD47 fusion protien with a thiol tag may be prepared and is PEGylated with PEG-maleimide or other PEG derivatives that react with thio tag specificlly to obtain the desired structure.

### P Moiety

In some embodiments of present invention, the B-P moiety may be derived from a PEG of the formula:

B—O—(CH₂CH₂O)ₙCH₂(CH₂)ₘF

wherein:

n is an integer from about 10 to 2300 to preferably provide polymer having a total molecule weight of from 3000 to 40000 or greater if desired. B is a H or a low molecule weight alkyl group, non-limiting examples of B include methyl, ethyl, isopropyl, propyl, and butyl. m is an integer selected from 0 to 10. F is a terminal functional group such as hydroxyl, carboxyl, thiol, halide, amino group etc. which is capable of being functionalized, activated and/or conjugated to a trifunctional small molecule compound.

In another embodiment of present invention, the B-P moiety may comprise an branched PEG. The branched P moiety may be derived from a compound of the formula:

(B-PEG)ₘL—Sᵢ—Fᵢ

wherein:

PEG is polyethylene glycol. m is an integer greater than 1 to preferably provide polymer having a total molecule weight of from 3000 to 40000 or greater if desired. B is a H or a methyl or other low molecule weight alkyl group. L is a functional linkage moiety to which two or more PEGs are attached. Examples of such linkage moiety are: any amino acid such as glycine, alanine, lysine, or 1,3-diamino-2-propanol, triethanolamine, any 5 or 6 member aromatic ring or aliphatic rings with more than two functional groups attached etc. S is any non-cleavable spacer. F is a terminal functional group such as hydroxyl, carboxyl, thiol, amino group, etc. i is 0 or 1.

In case that i equals to 0, the formula is:

(B-PEG)ₘL

wherein PEG, m, B or L have the same definitions as described above.

The compound of the present invetion may comprise, or the method of the present invention may be carried out with alternative polymeric substances such as dextrans, carbohydrate-base polymers, polyalkylene oxide, polyvinyl alcohols or other similar non-immunogenic polymers, the terminal groups of which are capable of being functionalized or activated to be converted to heterobifunctional groups. The foregoing list is merely illustrative and not intended to restrict the type of non-immunogenic polymer suitable for use herein.

### Trifunctional Linker T

T represents a trifunctional linker, connecting with P, (L¹)ₐ and (L²)_{b}. T may be derived from molecules with any combination of three functional groups, non-limiting examples of which include hydroxyl, amino, hydrazinyl, carboxyl, thiol, and halide. The functional groups may be the same or different in a trifunctional linker. One or more of the functional groups of the trifunctional linker may also be converted into one or more other groups before or after the reaction between T and the reaction partners. For example, a hydroxyl group may be converted into a mesylate or a tosylate group. A halide may be displaced with an azido group. An acid functional group of T may be converted to an alkyne function group by coupling with an amino group bearing a terminal alkyne.

In some embodiments, T may be derived from a natural or unnatural amino acid selected from the group consisting of cysteine, lysine, asparagine, aspartic, glutamic acid, glutamine, histidine, serine, threonine, tryptophan, tyrosine or genetically-encoded alkene lysine (such as N6-(hex-5-enoyl)-L-lysine), 2-Amino-8-oxononanoic acid, m- or p-acetyl-phenylalanine, amino acid bearing a f3-diketone side chain (such as 2-amino-3-(4-(3-oxobutanoyl)phenyl)propanoic acid), (S)-2-amino-6-(((1R,2R)-2-azidocyclopentyloxy)carbonylamino) hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ϵ}-Acryloyl-1-lysine, N-ϵ-5-norbornene-2-yloxycarbonyl-1-lysine, N-ϵ-(Cyclooct-2-yn-1-yloxy)carbonyl)-L-lysine, N-ϵ-(2-(Cyclooct-2-yn-1-yloxy)ethyl) carbonyl-L-lysine, and genetically encoded tetrazine amino acid (such as 4-(6-methyl-s-tetrazin-3-yl)aminophenylalanine).

In exemplary embodiments, T is derived from cysteine, lysine, 1,3-diamino-2-propanol, or triethanolamine. One or more of the functional groups on these molecules may be protected for selective reactions. In some embodiments, T is derived from a BOC-protected lysine. In other embodiments, T is derived from a cysteine.

Bifunctional Linker L¹ and L²

Both linker L¹ and L² comprises linker chains, internal linkages and/or terminal linkages. Linker chains may be independently selected from an amino acid or a peptide having 2 to 50 amino acid residues or —(CH₂)ₐC(O)NR¹(CH₂)_{b}—, —(CH₂)ₐO(CH₂CH₂O)_{c}—, —(CH₂)ₐheterocyclyl-, —(CH₂)ₐC(O)—, and —(CH₂)ₐNR¹—, —CR¹═N—O—, —CR¹═N—NR²—CO—, —N═—CO—, —S—S—, wherein a, b, and c are each an integer selected from 0 to 25 with all subunits included; and R¹ and R² independently represent hydrogen or a C1-C10 alkyl.

Heterocyclyl linkage group of linker L¹ and L² (whether it is at internal position or at terminal position) may be derived from a maleimido-based moiety. Non-limiting examples of suitable precursors include N-succinimidyl 4-(maleimidomethyl) cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), ϵ-maleimidcaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p- maleimidophenyl)isocyanate (PMPI).

Alternatively, the heterocyclyl linkage group of the linker L¹ and L² may be tetrazolyl, trans-cyclooctene, azide or strianed alkyne. The heterocyclyl triazolyl linkages, for example, may be formed from conjugations of two different linker moieties: azide and strianed alkyne. Thus, the heterocyclyl group also serve as a linkage point.

In some embodiments, (L¹)ₐ and/or (L²)_{b} comprises:

X¹—(CH₂)ₐC(O)NR¹(CH₂)_{b}O(CH₂CH₂O)_{c}(CH₂)_{d}C(O)—, or

X³—(CH₂)ₐC(O)NR¹(CH₂)_{b}O(CH₂CH₂O)_{c}(CH₂)_{d}X² (CH₂)eN R²,

wherein X¹, X² and X³ may be the same or different and independently represent a heterocyclyl group;

a, b, c, d and e are each an integer selected from 1-25; and

R¹ and R² independently represent hydrogen or a C1-C10 alkyl.

In some embodiments, X¹ and/or X³ is derived from a maleimido-based moiety. In some embodiments, X² represents a triazolyl group. In some embodiments, R¹ and R² each represent a hydrogen. In some embodiments, a, b, c, d and e are each independently selected from 0, 1, 2, 3, 4, 5, 6, 7, 8, 9, and 10.

In some other non-limiting exemplary embodiments, each linker unit L¹ and L² may also be derived from a haloacetyl-based moiety selected from N-succinimidyl-4-(iodoacetyl)-aminobenzoate (SIAB), N-succinimidyl iodoacetate (SIA), N-succinimidyl bromoacetate (SBA), or N-succinimidyl 3-(bromoacetamido)propionate (SBAP).

### Linkage Group

Different moieties of the conjugates of the present invention may be connected via various chemical linkages. Examples include but are not limited to amide, ester, disulfide, ether, amino, carbamate, hydrazine, thioether, and carbonate.

For instance, the terminal hydroxyl group of a PEG moiety (P) may be activated and then coupled with lysine (T) to provide a desirable linkage point between P and T of Formula Ia or Ib. Meanwhile, the linkage group between T and L¹ or L² may be an amide resulting from the reaction of the amino group of a linker L¹ or L² with the carboxyl group of Lysine (T). Alternatively, the linkage group between T and L¹ or L² may be an amide resulting from the reaction of the amino group of T with activated carboxyl group of a linker L¹ or L² . Depending on the desirable characteristics of the conjugate, suitable linkage groups may also be incorporated between the antibody moiety (A) and the adjacent linker (L² or L²) and between or within individual linkers of L¹ or L².

In some embodiments, the linkage group between different moieties of the conjugates may be derived from coupling of a pair of functional groups which bear inherent chemical affinity and selectivity for each other. These types of coupling or ring formation allow for site-specific conjugation for the introduction of a particular protein or antibody moiety. Non-limiting examples of these functional groups that lead to site-specific conjugation include thiol, maleimide, 2′-pyridyldithio variant, aromatic or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, and triarylphosphine.

### Synthesis

In some embodiments, the processes of synthesis are examplified as follows.

Once the desired size and shape of PEG (linear or branched) has been selected, the terminal functional group of PEG such as hydroxyl or carboxyl group etc. is converted to terminal branched heterobifunctional groups using any art-recognized process. Broadly stated, the terminal branched heterobifunctional PEG such as terminal branched heterobifunctional PEG-Lys(maleimide)-alkyne is prepared by activating terminal hydroxyl or carboxyl group of the PEG with N-Hydroxysuccinimide using reagents such as Di(N-succinimidyl) carbonate (DSC), triphosgene etc. in case of terminal hydroxyl group or coupling reagents such as N,N′-Diisopropylcarbodiimide (DIPC), 1-Ethyl-3-(3-dimethylaminopropyl)carbodiimide (EDC) etc. in case of terminal carboxyl group in the presence of base such as 4-Dimethylaminopyridine (DMAP), pyridine etc. to form activated PEG. Next, the activated PEG is reacted with a trifunctional small molecule such as lysine derivative H-Lys(Boc)-OH in the presence of base such as Diisopropylamine (DIPE) to form a terminal branched heterobifunctional PEG with a free carboxyl group and a Boc protected amino group PEG-Lys(Boc)-COOH.

As will be appreciated by those of ordinary skill, other known terminal functional groups of PEG such as halide, amino, thiol group etc. and other known tri-functional small molecules may be used as alternatives for the same purpose if desired. Examples of tri-functional small molecules include the molecules containing any combination of three functional groups (NH₂, NHNH₂, COOH, OH, C═OX, N═C═X, SH, anhydride, alkyl halide, maleimide, C═C, C≡C etc. wherein X is a halide) or their protected version. Treatment of the terminal branched Boc amino/carboxyl heterobifunctional PEG (PEG-Lys(Boc)-COOH) with an acid such as trifluoroacetic acid (TFA) gives the terminal branched amine/carboxyl heterobifunctional PEG (PEG-Lys-COOH). The terminal branched amine/carboxyl heterobifunctional PEG is then converted to a terminal branched maleimide/carboxyl heterobifunctional PEG (PEG-Lys(maleimide)-COOH) by reacting with a bifunctional small molecule spacer that has a maleimide group such as NHS-PEG2-Maleimide. The target terminal branched maleimide/alkyne heterobifunctional PEG (PEG-Lys(maleimide)-alkyne) is obtained by coupling the terminal branched maleimide/carboxyl heterobifunctional PEG with another bifunctional small molecule spacer that has an alkyne group such as 1-amino-3-butyne or NH2-DBCO. This terminal branched maleimide/alkyne heterobifunctional PEG (PEG-Lys(maleimide)-alkyne) is allowed to and capable of site-specific conjugations with two different proteins or antibodies consecutively, e.g. one with a thiol tagged single chain antibody such as anti-CD3 and the other with an azide tagged single chain antibody such as anti-CD47.

In some embodiments, the two single chain antibody (SCA) fragments, anti-CD3 (SCACD3) and anti-CD47 (SCACD47) may be generated using various technology known in the art. In one example, nucleotide sequences encoding the antibody such as anti-CD3 VH-VL and anti-CD47 VL-VH are synthesized and cloned into a GS (glutamate synthase) gene containing expression vector, and the resultant expression construct bearing a signal peptide for secretion are transfected into the GS gene knockout CHO cell line. High expression cell pools or clones are screened and the proteins are expressed thereafter. To facilitate the subsequent conjugation, a site specific functional group such as thiol is inserted through recombinant DNA technology into the linker between VH and VL (Yang, K. et al. 2003, Protein Eng 16 (10), p761-770) of the single chain antibodies. Pure SCA is obtained via chromatographic process. As will be appreciated by those of ordinary skill, other known site specific functional groups may also be inserted through recombinant DNA technology into the linker between VH and VL of the SCA as alternatives for the same purpose if desired.

In some embodiments, to prepare PEGylated single chain bispecific antibody, the terminal branched alkyne/maleimide heterobifunctional PEG (PEG-Lys(maleimide)-alkyne) is reacted site specifically with a free thiol functional group of SCACD3 that is genetically inserted, resulting in PEG(SCACD3)-alkyne, while SCACD47 is conjugated site specifically with a small molecule azide/maleimide bifunctional linker, resulting in azide-SCACD47. Purified azide-SCADCD47 and purified PEG(SCACD3)-alkyne are reacted site specifically through an azide-alkyne clicking chemistry to form a target PEGylated single chain bispecific antibody PEG (SCACD3)SCACD47.

In addition to thiol/maleimide and azide/alkyne site specific conjugation group pair used in this invention, as will be appreciated by those of ordinary skill, other known pairs of site-specific conjugation groups, such as thiol/2′-pyridyldithio pair; thiol/sulfone pair; DBCO/azide pair; trans-cyclooctenes/tetrazines pair; carbonyl/hydrazide pair; carbonyl/oxime pair; azide/triarylphosphine pair; potassium acyltrifluoroborates/O-carbamoylhydroxylamines pair, may be similarly designed and used as alternatives for the same purpose if desired. The foregoing list of site-specific conjugation group pairs is merely illustrative and not intended to restrict the type of site-specific conjugation group pairs suitable for use herein.

Alternatively, a thio tagged single chain fusion bispecific antibody such as anti-CD3-Peptide-anti-CD47 with a thio tag may be conjugated directly with PEG-maleimide to arrive target structure if desired.

### Antibodies

The invention disclosed herein involves antibodies. As used herein, “antibody” is used in the broadest sense so long as they exhibit the desired biological activity. The bi-specific molecules of the present invention may be made using antibody fragments from any expression system (E. coli, yeast, drosophila or mammalian cell expression system). The antibody fragments bind to human CD3 or CD47. Preferably the antibody fragments are derived from human antibodies, although the antibodies may also be, for example, murine antibodies, chimeric antibodies, humanized antibodies, or a combination thereof.

### Fragment

In certain embodiments, an antibody provided herein is an antibody fragment. Antibody fragments include, but are not limited to, Fab, Fab′, Fab′-SH, F(ab′)₂, Fv, and scFv fragments, and other fragments described below, e.g., diabodies, triabodies tetrabodies, nanobody and single-domain antibodies.

Various techniques have been developed for the production of antibody fragments. Traditionally, antibody fragments may be obtained via proteolytic digestion of full length antibodies (see, e.g., Morimoto, K., et al., 1992, J. Biochem. Biophys. Meth. 24, p107-117, Brennan, M., et al., 1985, Science 229, p81-83).

Antibody fragments may also be produced directly by recombinant means. Fab, Fv and scFv antibody fragments may all be expressed in and secreted from e.g. E. coli, thus, allowing the facile production of large amounts of these fragments. Antibody fragments may be isolated from antibody phage libraries according to standard procedures. Alternatively, Fab′-SH fragments may be directly recovered from E. coli. (Carter, P., et al., 1992, Bio/Technology 10, p163-167). Mammalian cell systems may be also used to express and, if desired, secrete antibody fragments.

### Chimeric and Humanized Antibodies

In certain embodiments, an antibody provided herein is a chimeric antibody. In some embodiments, a chimeric antibody comprises a non-human variable region. In some other embodiments, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Generally, a humanized antibody comprises one or more variable domains in which HVRs, e.g., CDRs, (or portions thereof) are derived from a non-human antibody, and FRs (or portions thereof) are derived from human antibody sequences.

Humanized antibodies and methods of making the same have been described in references such as U.S. Pat. Nos. 5,821,337, 7,527,791, 6,982,321, and 7,087,409, the entire disclosure of all of these patents are herein incorporated by reference.

### Human Antibodies

In certain embodiments, an antibody provided herein is a human antibody. Human antibodies may be produced using various techniques known in the art or using techniques described herein.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. Human variable regions from intact antibodies generated by such animals may be further modified, e.g., by combining with a different human constant region.

Human antibodies may also be made by hybridoma-based methods. Human myeloma and mouse-human heteromyeloma cell lines for the production of human monoclonal antibodies have been described. An exemplary procedure is provided in U.S. Pat. No. 7,189,826. Human hybridoma technology (Trioma technology) is also well known in the art.

Human antibodies may also be generated by isolating Fv clone variable domain sequences selected from human-derived phage display libraries.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are well known in the art.

In certain phage display methods, repertoires of VH and VL genes are separately cloned by polymerase chain reaction (PCR) and recombined randomly in phage libraries, which may then be screened for antigen-binding phage as known in the art. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire may be cloned (e.g., from human) to provide a single source of antibodies to a wide range of non-self and also self-antigens without any immunization as well known in the art. Finally, naive libraries may also be made synthetically by cloning unrearranged V-gene segments from stem cells, and using PCR primers containing random sequence to encode the highly variable CDR3 regions and to accomplish rearrangement in vitro, as well known in the art. Antibodies or antibody fragments isolated from human antibody libraries are considered human antibodies or human antibody fragments herein.

### Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution may be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved ADCC or CDC.

Accordingly, an antibody of the invention may comprise one or more conservative modifications of the CDRs, heavy chain variable region, or light variable regions described herein. The modification retains substantially the activity to of the parent peptide, polypeptide, or protein (such as those disclosed in this invention).

As used herein, the term “conservative modifications” refers to amino acid modifications that do not significantly affect or alter the binding characteristics of the antibody containing the amino acid sequence. Such conservative modifications include amino acid substitutions, additions and deletions. Modifications may be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include:
amino acids with basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine, tryptophan),
nonpolar side chains (e.g., alanine, valine, leucine, isoleucine, proline, phenylalanine, methionine),
beta-branched side chains (e.g., threonine, valine, isoleucine) and
aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques well known in the art. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Targets

The bispecific antibody (PEGylated anti-CD3Xanti-CD47) disclosed herein may be used in the preparation of medicaments for the treatment of e.g. an oncologic disease, a cardiovascular disease, an infectious disease.

In a preferred embodiment, a bispecific molecule is a conjugate of two antibodies or antigen-binding fragments thereof that specifically interact and show measurable affinities to CD3 and CD47, respectively. Each of the antibody binds to human CD3 or CD47 with a KD of 1×10⁻⁶ M or less, e.g., 1×10⁻⁷ M, 5×10⁻⁸ M, 1×10⁻⁸ M, 5×10⁻⁹ M, 1×10⁻⁹ M or less. Assays to evaluate the binding ability of the antibodies toward antigens are known in the art, including for example, ELISAs, Western blots and RIAs. The binding kinetics (e.g., binding affinity) of the antibodies also may be assessed by standard assays known in the art, such as by Biacore analysis.

In certain embodiments, the first or second recognition binding moiety comprises the heavy chain and light chain, or corresponding heavy chain and light chain CDR1, CDR2 and CDR3 of antibodies of interest. For example, each recognition binding moiety may be a single chain antibody Fv region (scFv). The CDR regions are delineated using the Kabat system (Kabat, E. A., et al. 1991, Sequences of Proteins of Immunological Interest, Fifth Edition, U.S. Department of Health and Human Services, NIH Publication No. 91-3242).

Listed below are examplary amino acid sequences of anti-CD3 scFv and anti-CD47 scFv.

Amino acid Sequence of SCACD3
(SEQ ID NO: 1):
DIKLQQSGAELARPGASVKMSCKTSGYTFTRYTMHWVKQR
PGQGLEWIGYINPSRGYTNYNQKFKDKATLTTDKSSSTAY
MQLSSLTSEDSAVYYCARYYDDHYCLDYWGQGTTLTVSSV
EGCGSGGSGGSGGSGGVDDIQLTQSPAIMSASPGEKVTMT
CRASSSVSYMNWYQQKSGTSPKRWIYDTSKVASGVPYRFS
GSGSGTSYSLTISSMEAEDAATYYCQQWSSNPLTFGAGTK
LELK
Amino acid Sequence of SCACD47
(SEQ ID NO: 2):
DIVMTQSPLSLPVTPGEPASISCRSSQSIVYSNGNTYLGW
YLQKPGQSPQLLIYKVSNRFSGVPDRFSGSGSGTDFTLKI
SRVEAEDVGVYYCFQGSHVPYTFGQGTKLEIKGGCSGGSG
GSGGSGGQVQLVQSGAEVKKPGASVKVSCKASGYTFTNYN
MHWVRQAPGQRLEWMGTIYPGNDDTSYNQKFKDRVTITAD
TSASTAYMELSSLRSEDTAVYYCARGGYRAMDYWGQGTLV
TVSS

### Modifications

In some embodiments, the V_{H} and/or V_{L} amino acid sequences of the antibodies may be 82% 85%, 90%, 95%, 96%, 97%, 98% or 99% homologous to the sequences set forth above and retain the corresponding antigen-binding activity and specificity.

In certain embodiments, an recognition binding moiety of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein, or conservative modifications thereof, and wherein the antibodies retain the desired functional properties.

A recognition binding moiety of the invention may be prepared using an antibody having one or more of the V_{H} and/or V_{L} sequences disclosed herein as starting material to engineer a modified antibody, which modified antibody may have altered properties from the starting antibody. An antibody may be engineered by modifying one or more residues within one or both variable regions (i.e., V_{H} and/or V_{L}), for example within one or more CDR regions and/or within one or more framework regions.

In still another embodiment, the glycosylation of an antibody may be modified. Glycosylation may be altered to, for example, increase or decrease the affinity of the antibody for antigen or an Fc receptor. Such carbohydrate modifications may be accomplished by, for example, altering one or more sites of glycosylation within the antibody sequence. For example, one or more amino acid substitutions may be made that result in elimination of one or more variable region framework glycosylation sites to thereby eliminate glycosylation at that site. Such an approach is described in further detail in U.S. Pat. Nos. 8,008,449 and 6,350,861.

### Compound

This invention provides a non-immunogenic polymer modified (e.g. PEGylated) anti-CD47Xanti-CD3 bispecific antibody.

As disclosed herein, anemia, thrombocytopenia and lymphopenia associated with the treatment of conventional anti-CD47 antibody are significantly improved with the present non-immunogenic polymer modified (e.g. PEGylated) anti-CD3Xanti-CD47 bi specific antibody due to the lack of Fc component usually seen in tranditional anti-CD47 antibodies as well as modification by the non-immunogenic polymer while the blood circulation half-life of the bispecific antibody is elongated. Moreover, disclosed bispecific antibody has further advantages of recruiting effector cells to kill the cancer cells in an efficient manner in addition to CD47/SIRP blockage. Furthermore, since traditional full length bispecific antibodies or their modified version are usually too large for deep penetration into the solid tumor tissues and traditional single chain bispecific antibodies or antibody fragments or their modified version have limited retention time in solid tumor tissues, it is of great challenging for treatment of solid tumor with such therapeutic antibodies. But the non-immunogenic polymer modified (e.g. PEGylated) anit-CD47Xanti-CD3 bispecific antibody disclosed herein has the ability to balance the size and circulation half life, and therefore may provide more effective treatment for solid tumors.

### Effectors

In some embodiments, the bi-specific compound or molecule can be further conjugated to one or more effector moieties, e.g. cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

### Compositions

The present invention also provides a composition, e.g., a pharmaceutical composition, containing bi-specific molecules of the present invention, optionally formulated together with a pharmaceutically acceptable carrier. For example, a pharmaceutical composition of the invention may comprise a bi-specific molecule that binds to both CD3 and CD47.

Therapeutic formulations of this invention may be prepared by mixing the bi-specific molecules having the desired degree of purity with optional physiologically acceptable carriers, excipients or stabilizers (Remington's Pharmaceutical Sciences 1980, 16th edition, Osol, A. Ed.), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine;monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS, or polyethylene glycol (PEG).

The formulation may also contain more than one active compound as necessary for the particular indication being treated, preferably those with complementary activities that do not adversely affect each other. For instance, the formulation may further comprise another antibody, cytotoxic agent, or a chemotherapeutic agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 1980, 16th edition, Osol, A. Ed. Sustained-release preparations may be prepared. Suitable examples of sustained- release preparations include semipermeable matrices of solid hydrophobic polymers containing the bi-specific molecules, which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(--)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies may be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pharmaceutical compositions of the invention may be administered in combination therapy, i.e., combined with other agents. Examples of therapeutic agents that may be used in combination therapy are described in greater detail below.

The formulations to be used for in vivo administration must be sterile. This may be readily accomplished by filtration through sterile filtration membranes. Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### Dosage

The amount of active ingredient which may be combined with a carrier material to produce a single dosage form will vary depending upon the subject being treated, and the particular mode of administration. The amount of active ingredient which may be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the multi-specific molecules of this invention, the dosage ranges from about 0.0001 to 100 mg/kg, and more usually 0.01 to 50 mg/kg, of the host body weight. For example dosages may be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration twice per week, once per week, once every two weeks, once every three weeks, once every four weeks, once a month, once every 3 months or once every three to 6 months. Preferred dosage regimens for multi-specific molecules of the invention include 1 mg/kg body weight or 3 mg/kg body weight via intravenous administration, with the multi-specific molecule being given using one of the following dosing schedules: (i) every four weeks for six dosages, then every three months; (ii) every three weeks; (iii) 3 mg/kg body weight once followed by 1 mg/kg body weight every three weeks.

Alternatively, bi-specific molecules may be administered as a sustained release formulation, in which case less frequent administration is required. Dosage and frequency vary depending on the half-life of the multi-specific molecules in the patient. In general, human antibodies show the longest half-life, followed by humanized antibodies, chimeric antibodies, and nonhuman antibodies. The dosage and frequency of administration may vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient may be administered a prophylactic regime. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a multi-specific molecule of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth or metastasis by at least about 20%, more preferably by at least about 40%, even more preferably by at least about 60%, and still more preferably by at least about 80% relative to untreated subjects. The ability of an agent or compound to inhibit tumor growth may be evaluated in an animal model system predictive of efficacy in human tumors. Alternatively, this property of a composition may be evaluated by examining the ability of the compound to inhibit, such inhibition in vitro by assays known to the skilled practitioner. A therapeutically effective amount of a therapeutic compound may decrease tumor size, metastasis, or otherwise ameliorate symptoms in a subject. One of ordinary skill in the art would be able to determine such amounts based on such factors as the subject's size, the severity of the subject's symptoms, and the particular composition or route of administration selected.

### Administration

A composition of the invention may be administered via one or more routes of administration using one or more of a variety of methods known in the art. As appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a bispecific molecule of the invention may be administered via a non-parenteral route, such as a topical, epidermal or mucosal route of administration, for example, intranasally, orally, vaginally, rectally, sublingually or topically.

The active compounds may be prepared with carriers that will protect the compound against rapid release, such as a controlled release formulation, including implants, transdermal patches, and microencapsulated delivery systems. Biodegradable, biocompatible polymers may be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, 1978, J. R. Robinson, ed., Marcel Dekker, Inc., New York.

Therapeutic compositions may be administered with medical devices known in the art. For example, a therapeutic composition of the invention may be administered with a needleless hypodermic injection device, such as the devices disclosed in U.S. Pat. Nos 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,4391,96, and 4,475,196. These patents are incorporated herein by reference. Many other such implants, delivery systems, and modules are known to those skilled in the art.

### Treatment Methods

In one aspect, the present invention relates to treatment of a subject in vivo using the above-described bispecific molecule such that growth and/or metastasis of cancerous tumors is inhibited. In one embodiment, the invention provides a method of inhibiting growth and/or restricting metastatic spread of tumor cells in a subject, comprising administering to the subject a therapeutically effective amount of a bispecific molecule. Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g. clear cell carcinoma), prostate cancer (e.g. hormone refractory prostate adenocarcinoma), colon cancer and lung cancer (e.g. non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the immune response.

The above treatment may also be combined with standard cancer treatments. For example, it may be effectively combined with chemotherapeutic regimes. In these instances, it may be possible to reduce the dose of chemotherapeutic reagent administered (Mokyr, M. et al. 1998, Cancer Research 58, p5301-5304).

Other antibodies which may be used to activate host immune responsiveness may be used in combination with the bispecific molecule of this invention. These include molecules targeting on the surface of dendritic cells which activate DC function and antigen presentation. For example, anti-CD40 antibodies are able to substitute effectively for T cell helper activity (Ridge, J. et al. 1998, Nature 393, p474-478) and may be used in conjunction with the multi-specific molecule of this invention (Ito, N. et al. 2000, Immunobiology 201, p527-40). Similarly, antibodies targeting T cell costimulatory molecules such as CTLA-4 (e.g., U.S. Pat. No. CD28 (Haan, J. et al. 2014, Immunology Letters 162, p103-112), OX-40 (Weinberg, A. et al. 2000, J Immunol 164, p2160-2169), 4-1BB (Melero, I. et al. 1997, Nature Medicine 3, p682-685), and ICOS (Hutloff, A. et al. 1999, Nature 397, p263-266) or antibodies targeting PD-1 (U.S. Pat. No. 8,008,449) PD-1L (U.S. Pat. Nos. 7,943,743 and 8,168,179) may also provide for increased levels of T cell activation. In another example, the multi-specific molecule of this invention may be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (eprtuzumab), AVASTIN (bevacizumab), and TARCEVA (erlotinib), and the like.

### Definitions of Terms

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred. The term C1-10 alkyl includes alkyl groups with 1, 2, 3, 4, 5, 6, 7, 8, 9 and 10 carbons. Similarly C1-25 alkyl includes all alkyls with 1 to 25 carbons. Exemplary alkyl groups include methyl, ethyl, isopropyl, n-butyl, n-pentyl, 2-methyl-1-butyl, 3-pentyl, 3- methyl-3-pentyl, and the like. As used herein, “alkyl” includes cycloalkyl when three or more carbon atoms are referenced. Unless otherwise noted, an alkyl may be substituted or un- substituted.

The term “function group” or “functional group” as used herein refers to a group that may be used, under normal conditions of organic synthesis, to form a covalent linkage between the entity to which it is attached and another entity, which typically bears a further functional group. A “bifuncational linker” refers to a linker with two functional groups forms two linkages via with other moieties of a conjugate.

The term “derivative” as used herein refers to a chemically-modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions.

The term “PEG” or “poly(ethylene glycol)” as used herein refers to poly(ethylene oxide). PEGs for use in the present invention typically comprise a structure of —(CH₂CH₂O)n-. PEGs may have a variety of molecular weights, structures or geometries. A PEG group may comprise a capping group that does not readily undergo chemical transformation under typical synthetic reaction conditions. Examples of capping groups include —OC1-25 alkyl or —OAryl.

The term “linker” or “linkage” as used herein refers to an atom or a collection of atoms used to link interconnecting moieties, such as an antibody and a polymer moiety. A linker may be cleavable or noncleavable. Cleavable linkers incorporate groups or moieties that may be cleaved under certain biological or chemical conditions. Examples include enzymatically cleavable disulfide linkers, 1,4- or 1,6-benzyl elimination, trimethyl lock system, bicine-based self cleavable system, acid-labile silyl ether linkers and other photo-labile linkers.

The term “linking group” or “linkage group” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linkage group.

The terms “peptide,” “polypeptide,” and “protein” are used herein interchangeably to describe the arrangement of amino acid residues in a polymer. A peptide, polypeptide, or protein may be composed of the standard 20 naturally occurring amino acid, in addition to rare amino acids and synthetic amino acid analogs. They may be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein may constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity may be measured by any appropriate standard method, for example, by column chromatography, polyacrylamide gel electrophoresis, or HPLC analysis. An isolated polypeptide/protein described in the invention may be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

As used herein, “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

As used herein, “antibody fragments”, may comprise a portion of an intact antibody, generally including the antigen binding and/or variable region of the intact antibody and/or the Fc region of an antibody which retains FcR binding capability. Examples of antibody fragments include linear antibodies; single-chain antibody molecules; and multispecific antibodies formed from antibody fragments. Preferably, the antibody fragments retain the entire constant region of an IgG heavy chain, and include an IgG light chain.

As used herein, the term “Fc fragment” or “Fc region” or “Fc” is used to define a C-terminal region of an immunoglobulin heavy chain.

The term “traditional antibody” is used herein to refer to whole length monoclonal antibody or its modified version.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256, p495-49′7, which is incorporated herein by reference, or may be made by recombinant DNA methods (see, e.g., U.S. Pat. No. 4,816,567, which is incorporated herein by reference). The monoclonal antibodies may also be isolated from phage antibody libraries using the techniques described in Clackson et al., 1991, Nature, 352, p624-628 and Marks et al., 1991, J Mol Biol, 222, p581-597, for example, each of which is incorporated herein by reference.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., 1984, Proc Natl Acad Sci USA, 81, p6851-6855; Neuberger et al., 1984, Nature, 312, p604-608; Takeda et al., 1985, Nature, 314, p452-454; International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., 1986, Nature, 321, p522-525 (1986); Riechmann et al., 1988, Nature, 332, p323-327; Presta, 2003, Curr Op Struct Biol, 13(4), p519-525; U.S. Pat. No. 5,225,539, each of which is incorporated herein by reference. “Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition must be “acceptable” also in the sense that it is compatible with the active ingredient and may be capable of stabilizing it. One or more solubilizing agents may be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g., Berge, S. M., et al. 1977, J. Pharm. Sci. 66, p1-19).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, see, e.g., 1994, Agnew Chem. Intl. Ed. Engl. 33(10), p183-186; dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5- FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethyl hy drazi de ; procarbazine; PSK®.; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapri stone, and toremifene (Fareston); and anti-androgens such as flutamide, nilutamide, bicalutamide, leuprolide, and goserelin; and pharmaceutically acceptable salts, acids or derivatives of any of the above.

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, in such cases, for example “about 1” may also mean from 0.5 to 1.4.

### EXAMPLES

### Example 1

### Preparation of 30kmPEG-Lys(maleimide)-DBCO

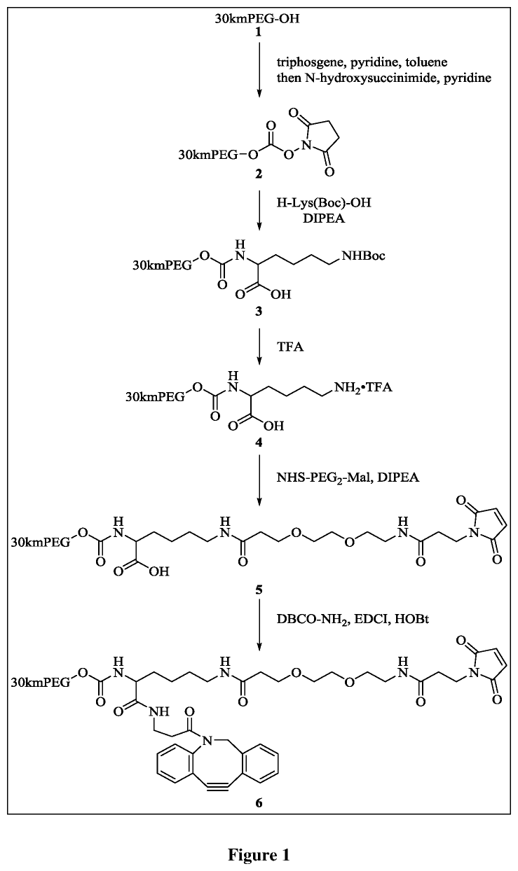

The schematic process of preparing 30kmPEG-Lys(maleimide)-DBCO is shown in FIG. 1. The specific steps are as follows.

Preparation of 30kmSC-PEG (Compound 2):

50 g of 30kmPEG-OH (MW=30000, 1 eq) was azeotroped for two hours with 720 mL of toluene to remove 150 mL toluene/water. After azeotroping, the solution was cooled to 45-50° C. 332 mg of triphosgene (0.67 eq.) was added to PEG followed by 263.6 mg of anhydrous pyridine (2 eq.). Reaction was stirred at 50° C. for 3 hours. 479.6 mg of N-hydroxysuccinimide (2.5 eq.) was then added followed by 329.6 g of anhydrous pyridine (2.5 eq.). The reaction mixture was stirred at 50° C. overnight under nitrogen. Pyridine salt was filtered. Solvent was removed with Rotavapor and the residue was recrystallized from 2-propanol. The isolated product was dried in vacuum oven at 40° C. to yield 46g of 30kmSC-PEG.

Preparation of 30kmPEG-Lys(Boc)-OH (Compound 3):

1107 mg of H-lys(boc)-OH (3eq.), 1939.5 mg of DIEA (10eq.) and 45 g of 30kmSCPEG (1 eq.) were mixed in 300 mL DMF and 450 ml DCM. The mixture was stirred at room temperature overnight. The insoluble materials were filtered off. The solvent was removed and the residue was recrystallized from 2-propanol. The isolated product was dried at 40° C. under vacuum to yield 42.5g of 30kmPEG-Lys(Boc)-OH.

Preparation of 30kmPEG-Lys-OH (Compound 4):

42 g of 30kmPEG-Lys(Boc)-OH (1 eq.) is treated with 630 mL of TFA/DCM (1:2) at room temperature for lhr. Solvent is removed under vacuum. The residue is recrystallized from ethyl ether/DCM. The isolated product is dried under vacuum at 40° C. to yield 39.9g of 30kmPEG-Lys-OH

Preparation of 30kmPEG-Lys(maleimide)-OH (Compound 5):

25.8 g of 30kmPEG-lys-OH (1 eq.) was dissolved in 258 mL of DCM and cooled to 0-5° C. 2.85 ml of DIEA (20 eq) was added followed by 1.1g of NHS-PEG2-Mal (3.0 eq) at 0-5° C. The mixture was stirred at room temperature overnight. Solvent was removed and the residue was recrystallized from 2-propanol. The isolated product was dried under vacuum to yield 24.4 g of 30kmPEG-Lys(maleimide)-OH.

Preparation of 30kmPEG-Lys(maleimide)-DBCO (Compound 6):

23.8 g of 30kmPEG-Lys(maleimide)-OH (1 eq) was dissolved in 238 mL of DCM at 0/5° C. followed by addition of 0.66g DBCO-NH2 (3.0 eq), 0.91g EDC (6.0 eq) and 0.96g HOBt (9 eq). The mixture was stirred at 0-5° C. for 2 hours. Then the reaction was left at room temperature overnight. Solvent was removed and the residue was recrystallized from 2-propanol. The isolated product was dried under vacuum at 40° C. to yield 22.1 g of 30kmPEG-Lys(Maleimide)-DBCO.

### Example 2

### Preparation of SCACD3 and SCACD47

Single chain antibody proteins are prepared accordingly as highlighted in Formula Ib, in which A1 is anti-CD3 (SCACD3) and A2 is anti-CD47 (SCACD47). Both proteins are prepared via recombinant DNA technology in Chinese hamster ovary (CHO) cells with GS knock out using pD2531nt-HDP expression vector containing GS gene (both the cell line and the vector are licensed from Horizon Discovery, Inc). DNAs encoding the first protein (SCACD3) and the second (SCACD47) are synthesized and cloned into pD2531nt-HDP expression vector and transfected to CHO-GS(-/-) cells. Stable cell lines with high production capacity were obtained by culturing the cells in medium containing GS inhibitor MSX without supplement of glutamine. The two scFvs produced by such cell lines were purified by Ni-chelating resin. Purified SCACD3 and SCACD47 are obtained via chromatographic process. The amino acid sequences of SCACD3 and SCACD47 are listed below.

Amino acid Sequence of SCACD3
(SEQ ID NO: 1):
DIKLQQSGAELARPGASVKMSCKTSGYTFTRYTMHWVKQR
PGQGLEWIGYINPSRGYTNYNQKFKDKATLTTDKSSSTAY
MQLSSLTSEDSAVYYCARYYDDHYCLDYWGQGTTLTVSSV
EGCGSGGSGGSGGSGGVDDIQLTQSPAIMSASPGEKVTMT
CRASSSVSYMNWYQQKSGTSPKRWIYDTSKVASGVPYRFS
GSGSGTSYSLTISSMEAEDAATYYCQQWSSNPLTFGAGTK
LELK
Amino acid Sequence of SCACD47
(SEQ ID NO: 2):
DIVMTQSPLSLPVTPGEPASISCRSSQSIVYSNGNTYLGW
YLQKPGQSPQLLIYKVSNRFSGVPDRFSGSGSGTDFTLKI
SRVEAEDVGVYYCFQGSHVPYTFGQGTKLEIKGGCSGGSG
GSGGSGGQVQLVQSGAEVKKPGASVKVSCKASGYTFTNYN
MHWVRQAPGQRLEWMGTIYPGNDDTSYNQKFKDRVTITAD
TSASTAYMELSSLRSEDTAVYYCARGGYRAMDYWGQGTLV
TVSS

### Example 3

### Preparation of 30kmPEG-(SCACD3)SCACD47

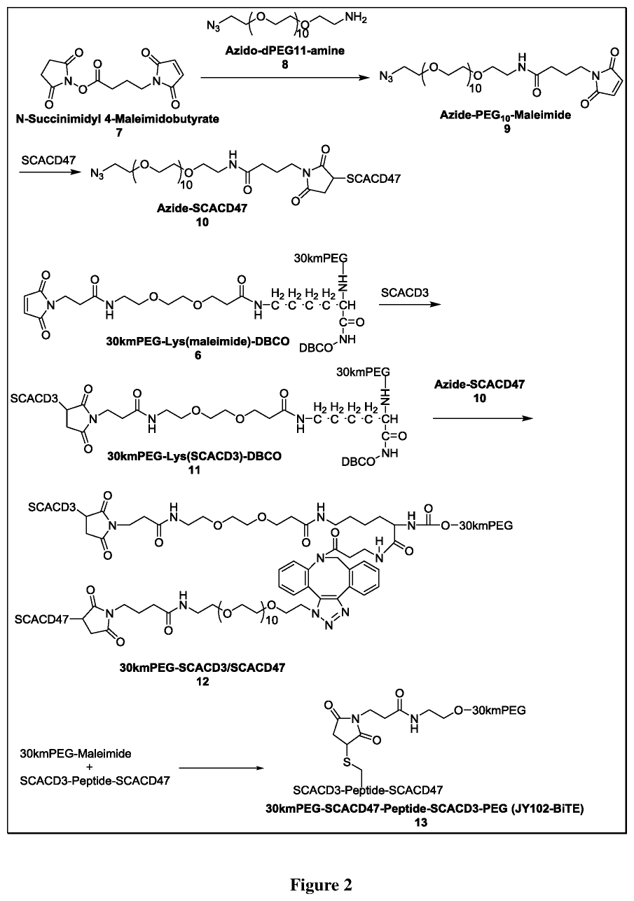

The schematic process of preparing 30kmPEG-(SCACD3)SCACD47 is shown in FIG. 2. The specific steps are as follows.

### Preparation of Azide-PEGio-Maleimide (Compound 9):

15 mg of N-Succinimidyl 4-Maleimidobutyrate (1 eq.) was reacted with 38mg of Azido-dPEG₁₀-amine (1.5 eq.) in 200 μl DMSO at room temperature for 45 min. Resulting compound azide-PEGio-Maleimide was used directly in next step without further purification. Preparation of Azide-SCACD47 (compound 10):

31 mg of SCACD47 (1 eq.) (1-5 mg/ml) was reduced by 2-8 mM of TCEP-HC1 in 100 mM phosphate, 1.5% PEG600, pH 6.8 for 30 min. The reduced SCADCD47 (1 eq.) was added to 200 μl of bifunctional linker Azide-PEGio-Maleimide (50 eq.). The mixture was vertexed and left on shaker at room temperature for 30 min to 2 hours. The reaction was quenched by 100 μl of 200 mM cysteine at room temperature for 10 min mixing with shaker. Excess linkers Azide-PEGio-Maleimide was removed by a catio exchange chromgrahy column (Poros™ XS, ThermoScientific, Bedford, MA, US) equilibrated with a 20 mM phosphate buffer, 1.5% Peg600 pH6.8. Azide-SCACD47 was gardiently eluted off. Fractions from Poros XS column were collected and analyzed by SDS-PAGE stained by SimplerBlueTM and SEC-HPLC. Based on the SDS-PAGE profile, desired compound Azide-SCACD47 fractions were pooled, concentrated to 5-10 mg/ml and stored in a refrigerator for further use.

Preparation of 30kmPEG-Lys(SCACD3)-DBCO (Compound 11):

24 mg of SCACD3 (1 eq.) (5-10 mg/ml) was reduced by 2-5 mM TCEP-HCl in 20 mM sodium phospahte, 1.5% PEG600, at pH 6.0 for 30 min. The reduced SCADCD3 (1 eq.) was mixed with 264 mg of 30KmPEG-Lys(Maleimide)-DBCO (10 eq.) in 100 mM Na phosphate, 1.5% PEG600, pH 6.8. The mixture was vertexed and left on shaker at room temperature for about 3 hours. The 30kmPEG-Lys(SCACD3)-DBCO was purified by a 20 mL CM Sepgharose Fast Flow (GE Healthcare) column pre-equilibrated with 20 mM sodium phosphate, 1.5% PEG600, pH6.0. After loading sample, the column was washed by 10 CV of equilibration buffer to wash off free PEGs, then eluted by 0.5 M NaCl. Fractions were collected and analyzed by SEC-HPLC and SDS-PAGE stained by SimplerBlue™ and iodine. Based on the SDS-PAGE profile, 30kmPEG-Lys(SCACD3)-DBCO was pooled and concentrated to 5-10 mg/ml.

Preparation of JY102 (30kmPEG-(SCACD3)SCACD47) (Compound 12):

Conjugation of 30kmPEG-Lys(SCACD3)-DBCO (compound 11) with Azide-SCACD47 (compound 10) was achieved by a clicking chemistry at 1:2 mole ratio in 20 mM phosphate, pH6.0 at room temperature for 2 hours while stirring. Purification of target PEGylated bispecific antibody 30kmPEG-(SCACD3)SCACD47 was performed first by a hydrophobic interaction chromatography (HIC) column (Phenyl HP (Gen Healthcare, NJ, US) with a 20 mM phosphate, (NH4)2SO4, pH6.3 buffer, followed by Poros™ X S(PorosTM XS, ThermoScientific, Bedford, MA, US) column. 30kmPEG-(SCACD3)SCACD47 was elueted from HIC column with gradient concentration of ammonium sulfate. Fractions from HIC were pooled and biuffer exchanged to a 20 mM phosphate, pH6.3, then polished by a PorosTM XS. All column chromatographic purifications were run by similar procedures described above. Fractions were collected and analyzed by SEC-HPCL and SDS-PAGE stained by SimplerBlue™ and iodine. Based on the SDS-PAGE profile, JY102 bispecific antibody product was pooled and concentrated to 5 mg/ml in 100 mM phosphate, pH6.0. The target compound was confirmed by SEC-HPLC and cell based activity assay. Purfied JY102 was concentrated to 5 mg/mL, then sterile filtered in a buffer of 10 mM acetate, 150 mM NaCl, pH4.7 and stored at 4° C. for further assay.

### Example 4

### Preparation of SCACD47/SCACD3 BiTE Fusion Protein

The fusion protein of two single chain antibodies SCACD47/SCACD3 BiTE was prepared using the method described in example 2, except that the amino acid sequence for the fusion protein is different. In addition, between SCACD47 and SCACD3, a 15 amino-acd peptide (GCGSGGSGGSGGSGG) was inserted, in which the cystein (C) was used for pegylation in subsquent procedures. The fusion protein produced was purified by Ni-chelating resin. Purified SCACD47-Peptide-SCACD3 was obtained via chromatographic process. The amino acid sequence of SCACD47-Peptide-SCACD3 fusion protein is listed below.

Amino acid Sequence of SCACD47/
SCACD3 BITE (SEQ ID NO: 3):
DIVMTQSPLSLPVTPGEPASISCRSSQSIVYSNGNTYLGW
YLQKPGQSPQLLIYKVSNRFSGVPDRFSGSGSGTDFTLKI
SR VEAEDVGVYYCFQGSHVPYTFGQGTKLEIKGGSGGSG
GSGGSGGQVQLVQSGAEVKKPGASVKVSCKASGYTFTNYN
MHWVRQAPGQRLEWMGTIYPGNDDTSYNQKFKDRVTITAD
TSASTAYMELSSLRSEDTAVYYCARGGYRAMDYWGQGTLV
TVSSGCGSGGSGGSGGSGGDIKLQQSGAELARPGASVKMS
CKTSGYTFTRYTMHWVKQRPGQGLEWIGYINPSRGYTNYN
QKFKDKATLTTDKSSSTAYMQLSSLTSEDSAVYYCARYYD
DHYCLDYWGQGTTLTVSSVEGGSGGSGGSGGSGGVDDIQL
TQSPAIMSASPGEK VTMTCRASSSVSYMNWYQQKSGTSP
KRWIYDTSKVASGVPYRFSGSGSGTSYSLTISSMEAEDAA
TYYCQQWSSNPLTFGAGTKLELKHHHHHH.

### Example 5

### Preparation of 30kmPEG-SCACD47-Peptide-SCACD3(JY102-BiTE) (FIG. 2)

Purified SCACD47-Peptide-SCACD3 fusion protein was concentrated to 5-10 mg/mL, buffer-exchanged to 100mM phosphate buffer at pH6.8, followed by treatment with 2-8 mM TCEP-HCl for 30 min at room temperature. 30KmPEG-Maleimide (10 eq.) in 100 mM Na phosphate was added followed by 1.5% PEG600. The reaction was run at pH 6.8 for 3 hour at room temperature while stirring. After reaction, excessive PEG was removed by a cation exchange chromatography column (Poros™ XS, Thermo Scientific, Bedford, MA, US) equilibrated with a 20 mM phosphate buffer, 1.5% PEG600 pH6.8. The product JY102-BiTE (compound 13) was eluted from column with gradient salt and fractions of JY102-BiTE was analysed by SDS-PAGE and SEC-HPLC. Purfied JY102-BiTE was concentrated to 5 mg/mL, then sterile filtered in a buffer of 10 mM acetate, 150 mM NaCl, pH4.7 and stored at 4° C.

### Example 6

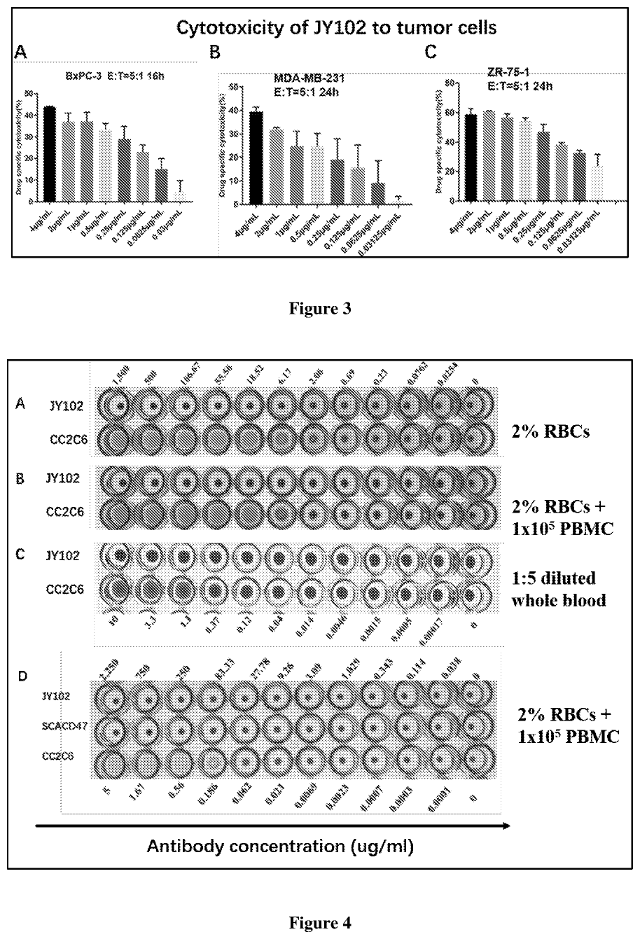

### In-Vitro T Cell Mediated Cytotoxicity (FIG. 3)

In vitro cytotoxicity assays were performed to evaluate and demonstrate the potency of PEGylated bispecific antibody compound 12 (JY102, 30kmPEG-SCACD3/SCACD47). The cytotoxicity of JY102 was determined using a color metric M T S assay.

In this assay, peripheral blood lymphocytes (PBMC) from healthy human donors were cultured and proliferated for 2-3 weeks following a T cell expansion protocol provided by a manufacturer's kit with some minor modifications (>90% are CD3+ T cells after cell proliferation). The T cell expanded PBMC were used as effector cells for the in vitro cytotoxicity assays. 4×10⁴ pancreatic cancer cells BxPC3 (or other CD47 positive cells MDA-MB-231, ZR-75-1) were seeded in a flat-bottom 96-well plate overnight to allow cells to adhere. In the next day, effector cells were washed, counted, and incubated with indicated doses of JY102 for 0.5 h at room temperature. Subsequently, effector cells together with JY102 were added to the target cells at 5:1 effector-to-target (E:T) ratios and incubated at 37° C. for 24 hours. 20u1 MTS (from Promega, Inc) was added into each well according to manufacturer's protocol. Absorbance at OD₄₉₀ nm was detected and the percentage of dead cells was calculated according the following formula:

Cytotoxicity %=1−(OD_{Experimental}−OD_{PBMC})/(OD_{Target}−OD_{medium}).

In this formula, OD_{Experimental} refers to the OD₄₉₀ of the wells containing JY102, effector cells and target at designed E:T ratio. OD_{PBMC} refers to the OD₄₉₀ of effector-cell-only with indicated JY102 doses but no target cells. OD_{target} refers to the OD₄₉₀ of target-cells-only with neither JY102 nor effector cells. OD_{medium} refers to the OD₄₉₀ of the equal volume of medium with none of JY102, effector cells or target cells.

As shown in FIG. 3, The cytotoxicity is drug specific, which has subtracted the background cytotoxicity to the targets mediated by the effector cells only (no JY102 was added). The result demonstrated that JY102 was potent in lysing CD47 expressing cells in the presence of effector T cells. The EC₅₀ value of JY102 (analyzed with the 4 parameter logistic non-linear regression model fit by GraphPad Prism 6) was 111.2 ng/ml to ZR75-1, indicating that the JY102 is significantly cytotoxic toward the target cells. In addition, the JY102 has shown dose-dependent killing effect toward the target cells.

It is noteworthy that the cytotoxicity of JY102 shown here is only effected through the mechanism of BiTE, because the effector cells used in the assay were T cell proliferated PBMC, which barely had any residual phagocytes. Better efficacy could be reasonably expected for JY102 when fresh PBMC that has normal level of phagocytes were used because JY102 also has CD47 blockage mechanism that could activate phagocytes to kill cancer cells.

### Example 7

### In Vitro Hemagglutination Experiment Demonstrating Extraordinary Safety of Compound 12 (JY102) to Red Blood Cells (FIG. 4)

Constant and high level CD47 functions as a self-marker of red blood cells (RBCs) for preventing been eliminated by phagocytes. With the aging of RBCs, cell surface CD47 levels gradually decrease, and RBCs would be eventually cleared through phagocytosis in the spleen (Burger, P., et al., 2012, offizielles Organ der Deutschen Gesellschaft fur Transfusionsmedizin and Immunhamatologie, 39(5), p348-352). Macrophages and other phagocytes rely on the presence or absence of CD47 to distinguish self or foreign for RBCs. CD47/SIRPa may represent a potential pathway for the control of hemolytic anemia. As the current CD47 blockades by anti-CD47 antibodies frequently generate anemia in clinic, we examined the effects of compound 12 (JY102) to RBCs by in vitro hemagglutination (HA) assay, which reveals whether the CD47 antibodies are bonded to RBCs and induce the formation of the HA followed by cell death (US20140140989 A1).

To perform HA assay, whole blood, red blood cells (RBC), and PBMCs were obtained from healthy donors, washed and re-suspended. The antibodies (JY102 or control anti-CD47 mAb CC2C6) at the indicated final concentrations as shown in FIG. 4 were added to RBCs in round bottom 96-well microplates and incubated for 2-4 h at 37° C. The results of HA formation could be observed with naked eyes. A diffused hazy pattern indicates HA whereas a small punctate circle in the well indicates no HA. In FIG. 4, panel A, B and C showed the concentrations of both JY102 (upper row of each panel) and CC2C6 (a purchased anti-CD47 known to induce HA) in 1:3 serial dilution, with JY102 starting at 1500 ug/ml, and CC2C6 starting at 10 ug/ml. In panel D, the starting concentration for the 1:3 serial dilution of both JY102 and the CD47 scFv (SCACD47) that is used to make JY102 was 2250 ug/ml, while the control CC2C6 starts at 5 ug/ml.

The results clearly demonstrated that, whether it is in 2% RBCs, 1:5 diluted whole blood or 2% RBC plus fresh isolated PBMC (10⁵), JY102 did not induce HA formation at high concentrations up to 1. 5mg/ml. Even at the highest concentration of 2.25 mg/ml, no HA was observed for both JY102 and the SCACD47. Yet, for the control antibody CC2C6, which is well known to induce HA, the results showed that at a low concentration of 0.12 ug/ml, CC2C6 already starts to induce obvious HA formation.

### Example 8

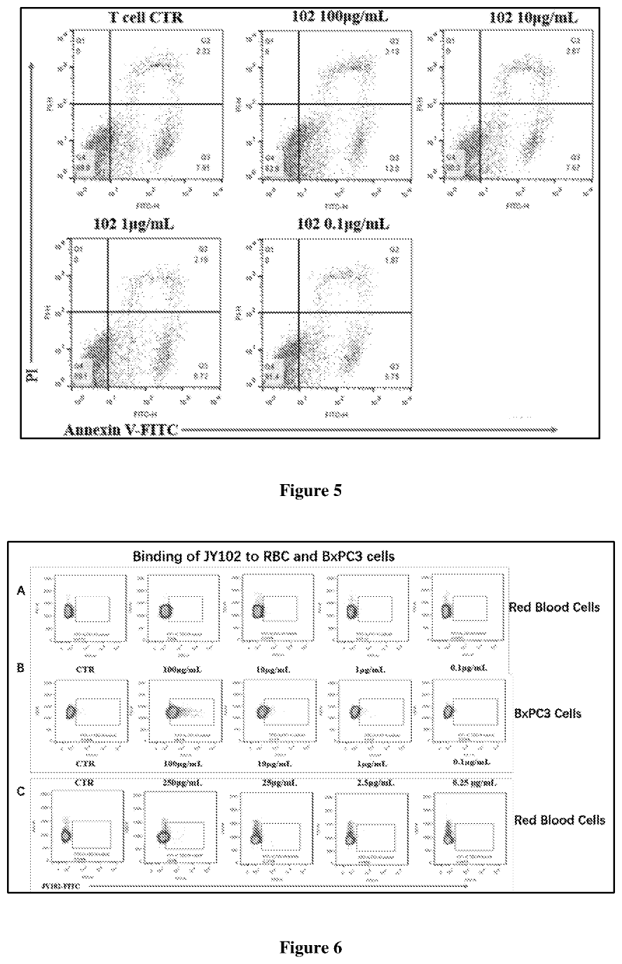

### Apoptosis Assay Demonstrating Extraordinary Safety of Compound 12 (JY102) to T Cells (FIG. 5)

T cells also express a high level of CD47. A frequently reported side effects of therapeutic CD47 blocking agents is lymphopenia, which includes reductions of B cells, T cells and NK cells. The purpose of this experiment is to examine whether compound 12 induces apoptosis of T cells. Although anti-CD47 antibody could enhance T cell immunity (Wu, L., et al., 2018, Oncolmmunology, 7(4), pe1397248), this effect may be impaired or abolished with substantial reduction of T cells in the blood cell population.

Annexin V-FITC and PI (4A BIOTECH, catalog #FXP018) were used in detecting early and late stage apoptotic cells, respectively. Proliferated PBMC from healthy human donors were used as T cell sources for this assay. Before the apoptosis assay, PBMC were cultured for 2-3 weeks following a modified T cell proliferation protocol, in which the dominant majority are T cells: at Day 14, CD19+ B cells were only 2.97%, while CD3+ T cells accounts for 98.8% in the proliferated population.

To perform the experiment, antibodies at the indicated final concentrations in FIG. 5 were added to proliferated PBMC (proliferated T cells) in round bottom 96-well microplates and incubated for 24 h at 37° C. Subsequently the cells were harvested and washed in cold phosphate-buffered saline (PBS), and then re-suspended in 1× Annexin-binding buffer at a concentration of 1×10⁶ cells/mL. To stain the apoptotic cells, 5 μL Annexin V and 51IL PI were added to each 100 μL of cell suspension. The cells were incubated at room temperature for 15 minutes in the dark. After the incubation, 400 μL 1× Annexin-binding buffer was added, mixed gently, and samples were kept on ice. The stained cells were analyzed by flow cytometry immediately.

The results demonstrate that under the high concentration of 100 ug/ml JY102, the apoptotic T cells (proliferated PBMC) were 16.13% (both Q2 and Q3), only 5.9% higher than the control T cells. For cells incubated with JY 102 at the concentration of lOug/ml, the apoptotic cells were 9.74%, which was almost the same as the control sample of 10.23%, which suggests that JY102 did not induce T cell apoptosis at this concentration. Although we have not performed head to head comparison with other CD47 blockades, the reported T cell apoptosis induced by 10 ug/ml Hu5F9-G4 was as high as 70%. Even for some improved version of anti-CD47 mAbs under commercial development (CN 111253488 A), the apoptosis rate was still as high as 45%.

### Example 9

### In Vitro Binding Assay Showing Differential Affinities of Compound 12 (JY102) to RBCs and BxPC3 Cells (FIG. 6)

As JY102 demonstrated extraordinary safety to both red blood cells and T cells while maintain the potent in inducing cytotoxicity of tumor cells, we were motivated to explore the mechanisms underneath. Binding assays of JY102 were thus performed to examine its affinities to target cells.

Again, RBC cells from healthy human donors or cultured BxPC3 cells were collected and resuspended to a concentration of approximately 5×10⁶ cells/ml in ice cold PBS, 3% BSA. 100 μl of cell suspension was used for each binding reaction and incubated with indicated doses of JY102-Alexa Fluor 488 for at least 30 min at room temperature (or 4° C. for the assay in panel C) in the dark. Then cells were washed 3 times by centrifugation at 400 g for 5 min and resuspended in ice cold PBS, 3% BSA. Each sample was subjected to flow cytometry analysis.

The assays were performed simultaneously for panels of A and B in FIG. 6. The results demonstrated that at the concentration of 10 ug/ml, JY102 stained RBCs were 0.551%, while JY102 bound BxPC3 cells were 22.1%, a 40-fold difference in binding. At the concentration of 100 ug/ml JY102, only 7.24% RBCs were stained positively, but stained BxPC3 cells were as high as 89.3%. At an even higher concentration of 250 ug/ml, the JY102 stained RBC was only 19.9% (panel C in FIG. 6). The huge significant differential affinity of JY102 to RBCs and tumor cells BxPC3 is probably due to the glycosylation pattern in the membrane of RBCs as previously reported (Meng, Q., et al., Front Neurosci, 2020. 14, p131). The structural modification of the CD47 scFv by PEG in JY102 may also contribute.

### Example 10

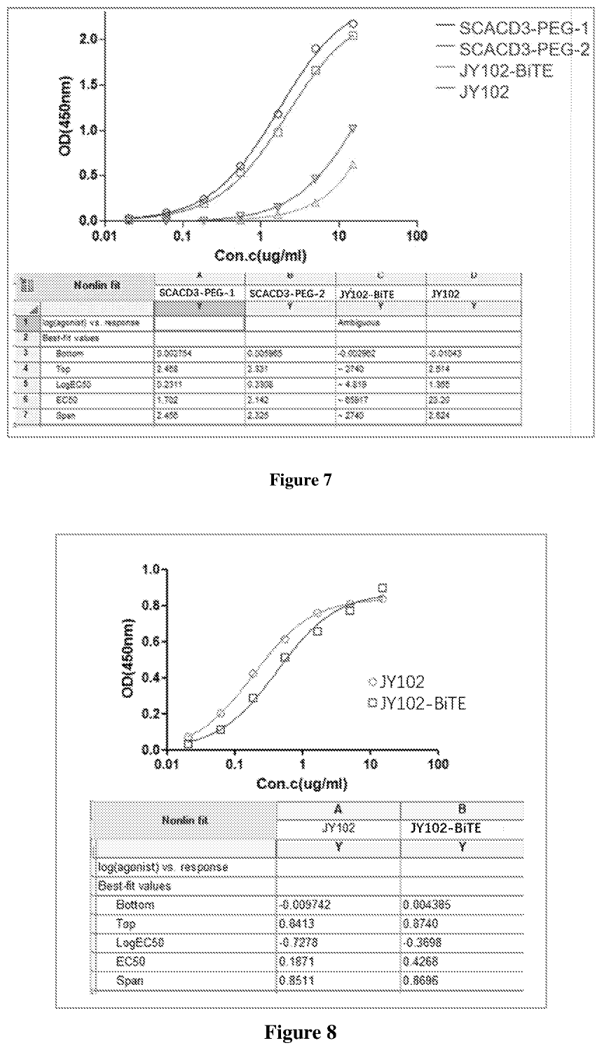

### Target Binding Assay for JY102 and JY102-BiTE (FIGS. 7 and 8)

Then the target binding capacity of JY102 (Compound 12) and JY102-BiTE (Compound 13) were tested using standard ELISA assays.

### Binding to Human CD3 Protein:

For CD3 target binding experiment, a 96-well plate was coated at 4° C. overnight with 200 ng/well Human CD3 epsilon & CD3 delta Heterodimer Protein (MALS verified) (Acro, cat: CDD-H52W1-50 ug). The next day, subsequent of 3 times of PBS wash, the coated plate was blocked with 1% BSA (in PB ST) at 37° C. for 2 hours followed with 3×PBS wash. Then JY102 or JY102-BiTE, or one of the two tested lots of SCACD3-PEG (compound 11) was added at 15 ug/ml for the first duplicate, and 1:3 serial dilutions of each compound were added sequentially afterwards, resulting in 7 different concentrations of JY102 (Lot #: 2020081202), JY102-BiTE (Lot #: 20201028), SCACD3-PEG-1 (Lot#: 20201020-1) or SCACD3-PEG-2 (Lot#: 20201020-2). After 37° C. incubation for lhr and subsequent 3×PBS wash, 5Ong anti-PEG (IBMS, Code: 6.3-PABG-B, Lot1-4) was added to each well and the plate was further incubated for lhr at 37° C. followed by 3×PBS wash. Then 50 ul diluted peroxides-conjugated streptavin (1:5000) was added to each well, and the plate was incubated at 37° C. for another lhr followed by 3×PBS wash. 100 ul TMB solution was added to each well and the plate was incubated at room temperature until desired signal was achieved. 100 μl STOP solution was added and absorbance at 450 nm was measured. Data was analyzed and EC₅₀ of human CD3 binding was calculated.

The result shown in FIG. 7 demonstrated that the ECso of CD3 target binding was very close for the two lots of SCACD3-PEG, while the bindings of JY102 and JY102-BiTE to CD3 were lower than SCACD3-PEG, which may be due to the steric hindrance effect of the additional scFv (SCACD47) added to compound 11 and is within the expectation. The reduced CD3 binding of JY102 and JY102-BiTE than SCACD3-PEG is desired because a high binding to CD3 may cause severe side effects in vivo, and a lower binding affinity would help avoid on-target off-tumor cytotoxicity and cytokine release by T cells as demonstrated in Her2xCD3 BsAbs (Junttila, T., et al., 2014, Cancer Res, 74(19):5561-71; Slaga, D., et al, 2018, Sci Transl Med, eaat5775).

Bingding to Human CD47 Protein:

In this experiment, the bindings of JY102 and JY102-BiTE to the target CD47 were examined. The ELISA method is the same as that described in the previous exoeriment of binding to CD3 protein, except that the coating reagent is Human CD47 Protein, Fc Tag (HPLC verified; Acro, cat:CD7-H5256-100ug), and the detection antibody is anti-SCACD3 (SZEB property developed by GenScript, clone 7E2G5E3, lot: C9193E180/DD2004650). The reagents for coating and detection together could measure the complete molecule of JY102 (Lot #: 2020081202) and JY102-BiTE (Lot #: 20201028).

As shown in FIG. 8, both JY102 and JY102-BiTE have very high affinity to human CD47 protein, and the binding affinity of JY102 to target (EC50: 187.1 ng/ml) is even higher than that of JY102-BiTE (EC50: 426.8 ng/ml).

### Example 11

### Phamacokinetcis Study of JY102

The pharmacokinetics of JY102 was determined after intravenous injection at a concentration of 1 mg/kg body weight into wild type C57BL/6 mice. Because the half life of PEGylated conjugates in rodents are typically in the range of 5 times shorter than in humans (US 2011/0112021 A1), it may require a half life of about 10 hours or longer in this study for possible weekly drug administration of JY102 in human.

Healthy wild type male C57BL/6 mice (body weight of about 25g, n=3) were used in this study. The mice were injected intravenously at the tail with a single injection of 1 mg/kg JY102. At various time points (Pre-dose, 3 min, 10 min, 30 min, 1, 2, 5, 24, 48 72 and 96 hours after injection), about 0.1 ml blood samples were taken by retro-orbital bleeding approach and placed into tubes containing sodium heparin. After standstill at room temperature for 30 min, the samples were centrifuged at 4° C. and the serum were collected and frozen at −80° C. until assayed.

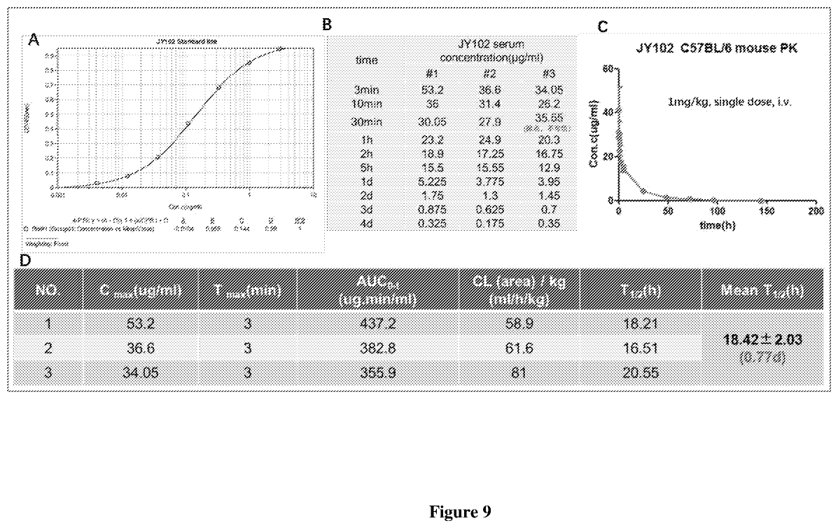

JY102 concentration in the serum samples at each time point was determined using ELISA assay. The coating reagent and detecting antibody are the same as in the experiment of CD47 target binding assay in example 10. A calibtation curve (FIG. 9 (A)) was established with JY102 standard samples with known concentrations, and the concentration ofJY102 in each serum sample was calculated from the calibtation curve. Then these serum concentrations were plotted against time as shown in FIG. 9 (B), and the PK data were thus obtained as shown in FIG. 9 (C) and (D).

The results in FIG. 9 (C) and (D) demonstrated that JY102 has a clerance half-life of 18.42 hrs in C57BL/6 mice, which can satisfy the weekly dosing schedule in clinical context.

It should be noted that the concentrations at the endpoint of our study (96 hrs) were still as high as 175 ng/ml to 350 ng/ml as shown in FIG. 9 (B), which is within working concentrations for JY102 in inducing cytotoxicity to tumor cells.

### Example 12

### Preliminary Toxicity Evalutaion of JY102 on PBMC Humanized Mice

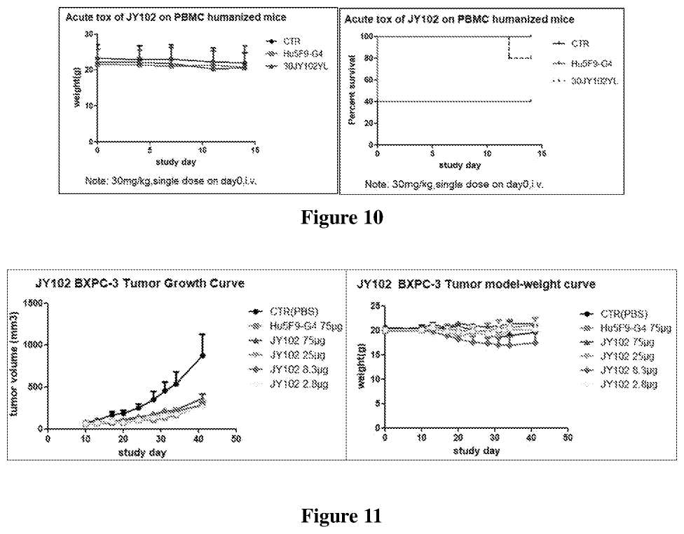

A preliminary acute toxicity study of JY102 was performed on PBMC humanzed mice. 5×10⁶ human PBMC were i.v. injected to 20 immune deficeint NPSG (NOD-Prkdc^{scid}II2rg^{null}) male mice (6 weeks) which had been used in previously described study (Xiao, J., et al., 2019, Cell death & disease 10, p777; Feng, M. et al. 2019, Nat Rev Cancer 19, p568-586; Matlung, H. L., 2017, Immunol Rev 276, p145-164). Three weeks later, each animal was blood sampled for flow cytometry analysis (using the same method descrined in example 8) on the fluoresent labled signals of human CD45, CD3 and CD14 to evaluate the PBMC recontruction status in vivo. 15 animals well reconstructed with human PBMC were divided into three groups for different treatments (n=5/group). The animals in JY102 group and Hu5F9-G4 were i.v. administered a single dose of 30mg/kg respectively, and the vehicle group animals received a single dose of PBS only. Daily observation and body weight measurement were performed after drug administration (FIG. 10).

From the results in FIG. 10, it can beseen that Hu5F9-G4 induced 60% death (3 of 5, n=5) and 20% near death (1 of 5) within 1 hour of i.v. injection of the drug, while JY102 did not generate death or near death, nor any observable injection induced toxicity other than the pale skin to one animal post injection. In the 2-week experimental span, JY102 was well tolerated, no abnormal activity or body weight fluctuation was observed. The single animal death from JY102 group on day 12 could be induced by PBMC rather than the JY102 toxicity.

Although more toxicity studies is warranted for furhter development of JY102, results from this preliminary experiment suggest the safety advantage of JY102 over Hu5F9-G4.

### Example 13

### JY102 Demonstrated Expected Efficacy In Inhibiting Tumor Growth

In order to evaluate efficacy of tumor inhibition of JY102, NPSG mouse (6 weeks old) was humanized by i.v. injection of 5×10 6 PBMC using the method described in example 12. One week after the PBMC injection, 1×10⁶ BxPC3 tumor cells were subsutaneously inoculated to each animal. The next day (set as day 0) after tumor cell injections, Hu5F9-G4 at the dose of and JY102 at the dose of 75 ug/animal, 25 ug/animal, 8.3 ug/animal and 2.8 ug/animal for each group (n=7) were administered. Subsequent doses were performed with JY102 administered at the frequency of every other day, and the Hu5F9-G4 group dosed every 10 days. The vehicle group animals received PBS treatment only. Tumor size and body weight were measured twice a week. The results are summarized in FIG. 11.

Comparing to the control group, all treatments inhibited BxPC3 tumor growth significantly (p<0.05 for control vs the any of the 4 treatment groups) . At the endpoint of the experiment, the tumor size of the control group was 536.1±326.6 mm³, and the tumor size of the smallest dosage (2.8 ug/animal or 0.014 mg/kg) group was 174.9±51.4 mm³ (67.4% inhibition). It is noteworthy that there is no significant efficacy difference among the treatment groups of JY102 (p>0.05) , which suggests that the experiment has not reached its lowest dosage of efficacy yet. Although Hu5F9-G4 also inhibited tumor growth by 69.1%, the dosage (75 ug/animal or 3.5 mg/kg) is more than 23 fold of the smallest JY102 dosage. As this experiment was performed on a PBMC humanized mice, unpredictable PBMC toxicity may existed, which propbably cause animal death in control group observed and body weight decrease in JY102 (8.3 ug) treatment group.

1. A compound having Formula Ib: 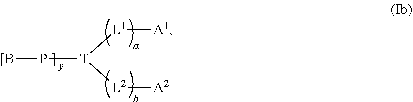
wherein:
P is a non-immunogenic polymer;
B is H or a capping group selected from C₁₋₁₀ alkyl and aryl, wherein one or more carbons of said alkyl or aryl is optionally replaced with a heteroatom;
One of A¹ and A² is an anti-CD3 antibody or an antigen binding fragment thereof, and the other is an anti-CD47 antibody or antigen binding fragment thereof, wherein the anti-CD3 antibody and the anti-CD47 antibody lack a functional Fc region;
L¹ and L² are each independently a bifunctional linker or a peptide;
a and b are each independently an integer selected from 1-10;
y is an integer selected from 1-10;
and
T is a trifunctional linker moiety comprising two linkages for (L¹)ₐ-A¹ and (L²)_{b}-A² and one linkage for P.

2. (canceled)

3. The compound of claim 1, wherein the anti-CD3 antibody and the anti-CD47 antibody are each independently selected from a Fab, a single chain antibody, and a nanobody (a single domain antibody).

4. The compound of any of claim 1, wherein the two linkages of T for (L¹)ₐ-A¹ and (L²)_{b}-A², the linkage between (L¹)ₐ and A¹, the linkage between (L²)_{b} and A² and the linkage within (L¹)ₐ or (L²)_{b} are each independently derived from functional groups selected from the group consisting of alkyl halide, acid halide, aldehyde, ketone, ester, anhydride, carboxylic acid, amide, amine, hydrazide, alkylhydrazines, hydroxy, epoxide, thiol, maleimide, 2-pyridyldithio varian, aromatic or vinyl sulfone, acrylate, bromo or iodo acetamide, azide, alkene, alkyne, dibenzocyclooctyl (DBCO), 2-amino-benzaldehyde or 2-amino-acetophenone group, hydrazide, oxime, potassium acyltrifluoroborate, O-carbamoylhydroxylamine, trans-cyclooctene, tetrazine and triarylphosphine.

5. The compound of any one of claim 1, wherein L¹ and L² each comprises a spacer independently selected from the group consisting of —(CH₂)ₘXY(CH₂)ₙ—, —X(CH₂)ₘO(CH₂CH₂O)ₚ(CH₂)ₙY—, —(CH₂)ₘX—Y(CH₂)n—, —(CH₂)ₘheterocyclyl-, —(CH₂)ₘX—, —X(CH₂)ₘY—, and an amino acid or a peptide having 2 to 50 amino acid residues; wherein m, n, and p in each instance are independently an integer ranging from 0 to 25; X and Y in each instance are independently selected from the group consisting of C(═O), CR₁R₂, NR₃, S, O, or Null, wherein R₁ and R₂ independently represent hydrogen, C₁₋₁₀ alkyl or (CH₂)₁₋₁₀C(═O), R₃ is H or a C₁₋₁₀ alkyl, and wherein the heterocyclyl is derived from an maleimido, strained alkenes and alkynes, azide or a tetrazolyl moiety.

6. The compound of any one of claim 1, wherein P comprises polyethylene glycol (PEG), dextrans, carbohydrate-base polymers, polyalkylene oxide, polyvinyl alcohols, hydroxypropyl-methacrylamide (HPMA), or a co-polymer thereof.

7. The compound of any one of claim 1, wherein P comprises PEG and B is methyl or a C₁₋₁₀ alkyl.

8. The compound of any one of claim 1, wherein P comprises PEG with a molecular weight ranging from 3000 Da to 80000 Da.

9. The compound of any one of claim 1, wherein P comprises a linear PEG or a branched PEG.

10. (canceled)

11. The compound of any one of claim 1, wherein the linkage of T to P is cleavable.

12. The compound of any one of claim 1, wherein the linkage of T to P is selected from the group consisting of amide, ester, carbamate, carbonate, imide, imine, hydrazones, sulfone, ether, thioether, thioester and disulfide.

13. The compound of any one of claim 1, wherein T is derived from a natural or unnatural amino acid selected from the group consisting of cysteine, lysine, asparagine, aspartic, glutamic acid, glutamine, histidine, serine, threonine, tryptophan, tyrosine or genetically-encoded alkene lysine (such as N6-(hex-5-enoyl)-L-lysine), 2-Amino-8-oxononanoic acid, m- or p-acetyl-phenylalanine, amino acid bearing a β-diketone side chain (such as 2-amino-3-(4-(3-oxobutanoyl)phenyl)propanoic acid) (S)-2-amino-6-(((1R,2R)-2-azidocyclopentyloxy)carbonylamino) hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ϵ}-Acryloyl-1-lysine, Nϵ-5-norbornene-2-yl oxycarb onyl-1-lysine, N-ϵ-(Cyclooct-2-yn-1-yloxy)carbonyl)-L-lysine, N-ϵ-(2-(Cyclooct-2-yn-1-yloxy)ethyl) carbonyl-L-lysine, and genetically encoded tetrazine amino acid (such as 4-(6-methyl-s-tetrazin-3-yl)aminophenylalanine).

14. The compound of claim 13, wherein T is derived from lysine or cysteine.

15. The compound of any one of claim 1, wherein P is derived from a PEG having a terminal maleimide or 2-pyridyldithio varian or aromatic sulfone or vinyl sulfone, T is derived from cysteine, and the linkage between P and T is a thioether or disulfide.

16. The compound of claim 15, wherein P is derived from a PEG having a terminal maleimide, and wherein (L¹)ₐ-T-(L²)_{b} is a peptide having 3-100 amino acid residues.

17. (canceled)

18. The compound of claim 1 having the following structure: 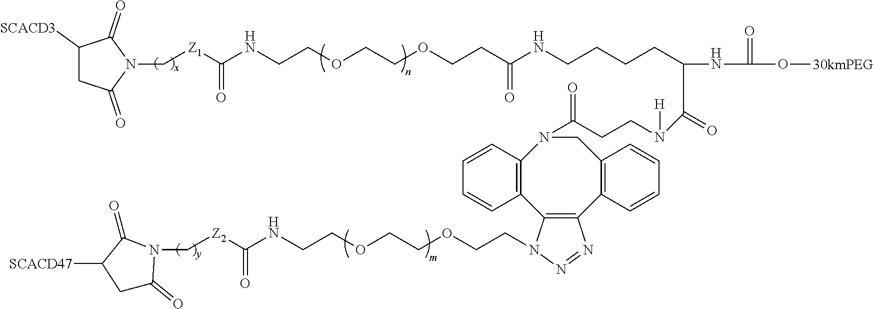
wherein SCACD3 is a single chain anti-CD3 antibody, SCACD47 is a single chain anti-CD47 antibody, Zi and Z2 are each independently selected from CH₂, a low-molecular-weight alkane, cyclohenane or its derivative, and n, m, x and y are each independently an integer selected from 0-50.

19. The compound of claim 1 having the following structure: 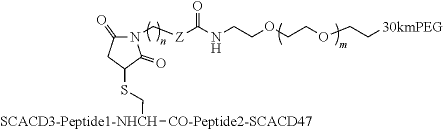
wherein SCACD3 is a single chain anti-CD3 antibody, SCACD47 is a single chain anti-CD47 antibody, Z is CH₂, a low-molecular-weight alkane, cyclohenane or its derivative, and n and m are each independently an integer selected from 0-50, and wherein peptide 1 and peptide 2 each independently comprises 2-50 amino acid residues.

20. The compound of claim 18, wherein the anti-CD3 antibody comprises the amino acid sequence set forth in SEQ ID NO: 1, and/or the anti-CD47 antibody comprises the amino acid sequence set forth in SEQ ID NO: 2.

21-23. (canceled)

24. A conjugate comprising the compound of claim 1 and one or more effector moieties conjugated to the compound, wherein the one or more effector moieties are selected from the group consiting of cytotoxic agents, chemotherapeutic agents, growth inhibitory agents, toxins, and radioactive isotopes.

25. (canceled)

26. A pharmaceutical composition comprising the compound of claim 1, and optionally a pharmaceutically acceptable carrier, excipient or stabilizer.

27. The pharmaceutical composition of claim 26, further comprising an additional therapeutic agent, wherein the additional therapeutic agent is selected from the group consisting of a cytotoxic agent, a chemotherapeutic agent, an antibody, an antibody drug conjugate, and a small molecule drug.

28. (canceled)

29. A method of treating a disease in a subject in need thereof comprising administering an effective amount of the compound of claim 1, wherein the disease is a cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer.

30. (canceled)

31. The compound of claim 19, wherein the anti-CD3 antibody comprises the amino acid sequence set forth in SEQ ID NO: 1, and/or the anti-CD47 antibody comprises the amino acid sequence set forth in SEQ ID NO: 2.


SEQUENCE LISTING (3 sequences)

SEQ ID NO: 1 - protein, 244 aa, Artificial Sequence
  Amino acid Sequence of SCACD3
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGCGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 244 aa, Artificial Sequence
  Amino acid Sequence of SCACD47
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKGGCSGGSGGSGGSGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 3 - protein, 507 aa, Artificial Sequence
  Amino acid Sequence of SCACD47/SCACD3 BiTE
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKGGSGGSGGSGGSGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGCGSGGSGGSGGSGGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH